(19) United States
(12) Patent Application Publication
(10) Pub. No.: US 2024/0018138 A1
(43) Pub. Date: Jan. 18, 2024

(54) PYRIDO[4,3-b]INDOLE DERIVATIVES AND THEIR USE AS PHARMACEUTICALS

(71) Applicant: Ventus Therapeutics U.S., Inc., Waltham, MA (US)

(72) Inventors: Lee Fader, Montreal (CA); Jason Burch, Montreal (CA); Miguel St-Onge, Montreal (CA); Stéphane Dorich, Montreal (CA)

(21) Appl. No.: 18/027,602

(22) PCT Filed: Sep. 23, 2021
(86) PCT No.: PCT/US2021/051670

§ 371 Date: Mar. 21, 2023

Related U.S. Application Data
(60) Provisional application No. 63/082,695, filed on Sep. 24, 2020.

(51) Int. Cl. C07D 471/04

(52) U.S. Cl. CPC C07D 471/04

(57) ABSTRACT

There is provided a compound of formula (I): or a pharmaceutically acceptable salt, solvate, ester or prodrug thereof. The compound of formula (I) can be useful for the treatment of a disease or condition for which a cGAS inhibitor is indicated. In some embodiments, the compound or its pharmaceutically acceptable salt, solvate, ester or prodrug thereof can be used for the treatment of an autoinflammation or an autoimmune disease such as systemic lupus erythematosus (SLE), Aicardi-Goutieres syndrome (AGS), nonalcoholic steatohepatitis (NASH), Age-dependent macular degeneration, Myocardial infarction, Acute pancreatitis, Ischemic stroke, Sporadic aortic aneurysm and dissection, Chronic lung disease, Inflammatory bowel disease, Parkinson's disease, Traumatic brain injury or Amyotrophic lateral sclerosis (ALS).

### CROSS-REFERENCE TO RELATED APPLICATIONS

The present application claims the priority benefit of U.S. Provisional Patent Application Ser. No. 63/082,695, filed Sep. 24, 2020, entitled PYRIDO[4,3-b]INDOLE DERIVATIVES AND THEIR USE AS PHARMACEUTICALS, incorporated by reference in its entirety herein.

### TECHNICAL FIELD

The technical field generally relates to compounds, compositions and their uses in the treatment of diseases and conditions in which inhibition of cyclic GMP-AMP synthase, also referred to as “cGAMP synthase” or simply “cGAS”, is indicated. For example, the application relates to pyrido[4,3-b]indole derivatives, to pharmaceutical compositions comprising the same, and to their use as cGAS inhibitors.

### BACKGROUND

cGAS is a predominant sensor for aberrant double stranded DNA (dsDNA) originating from pathogens, mislocalization or misprocessing of nuclear or mitochondrial cellular dsDNA. Binding of dsDNA to cGAS activates the synthesis of c[G(2′,5′)pA(3′,5′)p], a diffusible cyclic dinucleotide referred to as cGAMP, which activates the endoplasmic reticulum membrane-anchored adaptor protein, stimulator of interferon genes. The crucial role of cGAS in dsDNA sensing has been shown in different pathogenic bacteria, viruses, and retroviruses. Moreover, cGAS is involved in many other biological processes including cellular senescence and recognition of ruptured micronuclei in the surveillance of potential cancer cells. cGAS as also been shown to play a role in interferonopathies. Studies have shown that inhibition of cGAS may provide a therapeutic strategy for preventing autoinflammation and/or treating autoimmune diseases such as systemic lupus erythematosus (SLE) or Aicardi-Goutieres syndrome (AGS). The role of cGAS in cancer, diabetes, and immune disorders is well established. Recently, studies have also established that cGAS can promote the inflammatory and autophagy responses in Huntington disease.

Developing new inhibitors of cGAS is therefore important to provide further useful therapeutic agents for treating cGAS-related diseases. Small molecule inhibitors that are specific for cGAS would be of important value in treating diseases that arise from inappropriate cGAS activity.

### SUMMARY

According to one aspect, the present application relates to compound of formula (I):

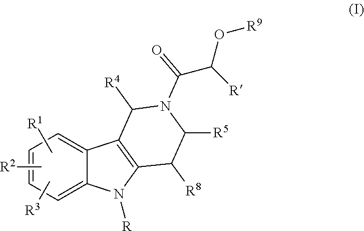

or a pharmaceutically acceptable salt, solvate, ester or prodrug thereof,
wherein:
R¹, R² and R³ independently represent hydrogen, halogen, CN, unsubstituted or substituted C₁-C₆ alkoxy, unsubstituted or substituted C₂-C₆ alkenyl, unsubstituted or substituted linear or branched C₁-C₆ alkyl, unsubstituted or substituted C₃-C₆ cycloalkyl or cycloalkenyl, unsubstituted or substituted C₆ aryl, unsubstituted or substituted 4 to 10 membered heterocycloalkyl being saturated or partially unsaturated, unsubstituted or substituted 4 to 10 membered heteroaryl, —NR¹⁰C(═O)R¹¹, —C(═O)NR¹²R¹³ or —CHR¹⁴R¹⁵,
   with at least one of R¹, R² and R³ being different than hydrogen;
R⁴, R⁵ and R⁸ independently represent H, unsubstituted or substituted linear or branched C₁-C₆ alkyl, —CH₂Ph, with at least one of R⁴ and R⁵ being different than hydrogen; or
   R⁵ and R⁸ are linked together to form a C₅-C₆ cycloalkyl;
R and R′ independently represent H or linear or branched C₁-C₃alkyl;
R⁹ is H or linear or branched C₁-C₃alkyl;
R¹⁰ is H or linear or branched C₁-C₃alkyl;
R¹¹ is unsubstituted or substituted linear or branched C₁-C₃alkyl,
R¹² is H or linear or branched C₁-C₃alkyl,
R¹³ is linear or branched C₁-C₃alkyl;
R¹⁴ is H or linear or branched C₁-C₃alkyl;
R¹⁵ is unsubstituted or substituted C₆ aryl, unsubstituted or substituted C₃-C₆ cycloalkyl or unsubstituted or substituted 5 to 6 membered heterocycloalkyl;
   wherein the heterocycloalkyl and heteroaryl groups include 1 to 3 heteroatoms independently selected from the group consisting of N, O and S;
   wherein, when any alkyl, alkoxy, alkenyl, cycloalkyl, cycloalkenyl, saturated or partially unsaturated heterocycloalkyl, aryl or heteroaryl groups are substituted, these groups are independently substituted with 1 to 3 substituents selected from the group consisting of halogen, hydroxy, methoxy, methyl, oxo (═O), CN, —NH₂, —NH(C₁-C₃alkyl), —N(C₁-C₃alkyl)₂, —NH(CO)CF₃, —CH₂OH, —CF₃, —CHF₂, —CH₂F, —SO₂NH(CH₂)₃OH and pyrazolyl.

In some embodiments, the compound can be a compound of formula (Ia) or (Ib) or a pharmaceutically acceptable salt, solvate, ester or prodrug thereof

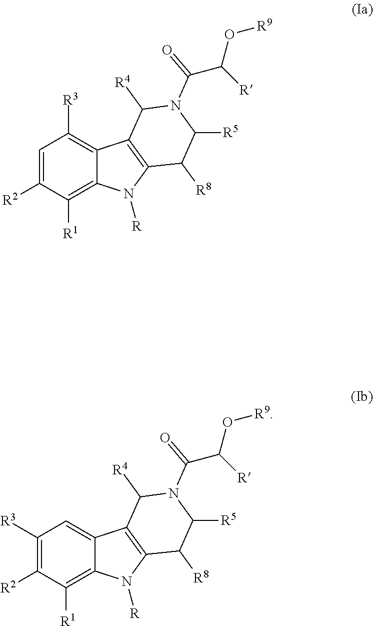

In some embodiments, the compound can be a compound of formula (Ic) or a pharmaceutically acceptable salt, solvate, ester or prodrug thereof

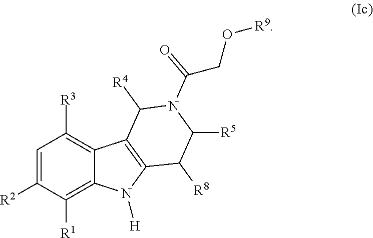

Another aspect relates to pharmaceutical compositions, comprising a compound as defined in the present application, together with a pharmaceutically acceptable carrier, diluent or excipient.

A further aspect relates to the use of a compound as defined in the present application, or such a compound for use, in the treatment or prevention of a disease or condition for which a cGAS inhibitor is indicated. Similarly, this aspect relates to the use of a compound of the present application in the manufacture of a medicament for the treatment or prevention of a disease or condition for which a cGAS inhibitor is indicated. This aspect also further relates to a method for treating a disease or condition for which a cGAS inhibitor is indicated, which comprises administering to a subject in need thereof, a therapeutically effective amount of a compound as herein defined. In one embodiment, the disease or condition for which a cGAS inhibitor is indicated can comprise Aicardi-Goutieres syndrome (AGS), systemic lupus erythematosus (SLE), nonalcoholic steatohepatitis (NASH), Age-dependent macular degeneration, Myocardial infarction, Acute pancreatitis, Ischemic stroke, Sporadic aortic aneurysm and dissection, Chronic lung disease, Inflammatory bowel disease, Parkinson's disease, Traumatic brain injury and Amyotrophic lateral sclerosis (ALS).

### DETAILED DESCRIPTION

### Definitions

All technical and scientific terms used herein have the same meaning as commonly understood by one ordinary skilled in the art to which the present technology pertains. For convenience, the meaning of certain terms and phrases used herein are provided below.

To the extent the definitions of terms in the publications, patents, and patent applications incorporated herein by reference are contrary to the definitions set forth in this specification, the definitions in this specification control. The section headings used herein are for organizational purposes only, and are not to be construed as limiting the subject matter disclosed.

The terminology used herein is for the purpose of describing particular embodiments only and is not intended to be limiting. It should be noted that, the singular forms “a”, “an”, and “the” include plural forms as well, unless the content clearly dictates otherwise. Thus, for example, reference to a composition containing “a compound” also contemplates a mixture of two or more compounds. It should also be noted that the term “or” is generally employed in its sense including “and/or” unless the content clearly dictates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the terms “compounds herein described”, “compounds of the present application” and equivalent expressions refer to compounds described in the present application, e.g., those encompassed by structural Formula I, optionally with reference to any of the applicable embodiments, and also includes exemplary compounds, for example, Compounds 1 to 140 of Table 1, as well as their pharmaceutically acceptable salts, solvates, esters, and prodrugs when applicable. When a zwitterionic form is possible, the compound may be drawn as its neutral form for practical purposes, but the compound is understood to also include its zwitterionic form. Embodiments herein may also exclude one or more of the compounds. Compounds may be identified either by their chemical structure or their chemical name. In a case where the chemical structure and chemical name would conflict, the chemical structure will prevail.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present description. Unless otherwise stated, all tautomeric forms of the compounds are within the scope of the present description. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of the present description. Such compounds are useful, for example, as analytical tools, as probes in biological assays, or as therapeutic agents in accordance with the present description.

Definitions of specific functional groups and chemical terms are described in more detail below.

The chemical structures herein are drawn according to the conventional standards known in the art. Thus, where an atom, such as a carbon atom, as drawn appears to have an unsatisfied valency, then that valency is assumed to be satisfied by a hydrogen atom even though that hydrogen atom is not necessarily explicitly drawn. Hydrogen atoms should be inferred to be part of the compound.

Abbreviations may also be used throughout the application, unless otherwise noted, such abbreviations are intended to have the meaning generally understood by the field. Examples of such abbreviations can include Me (methyl), Et (ethyl), Pr (propyl), i-Pr (isopropyl), Bu (butyl), t-Bu (tert-butyl), i-Bu (iso-butyl), s-Bu (sec-butyl), c-Bu (cyclobutyl), Ph (phenyl), Bn (benzyl), Bz (benzoyl), CBz or Cbz or Z (carbobenzyloxy), Boc or BOC (tert-butoxycarbonyl), and Su or Suc (succinimide).

The number of carbon atoms in a hydrocarbyl substituent can be indicated by the prefix “Cₓ-C_{y},” where x is the minimum and y is the maximum number of carbon atoms in the substituent. When reference is made to “x to y membered” heterocyclic ring (e.g., heterocycloalkyl, or heteroaryl), then x and y define respectively, the minimum and maximum number of atoms in the cycle, including carbons as well as heteroatom(s).

The prefix “halo” indicates that the substituent to which the prefix is attached is substituted with one or more independently selected halogen atoms. More specifically, the terms “halo” and “halogen” as used herein refer to an atom selected from fluorine (fluoro, —F), chlorine (chloro, —Cl), bromine (bromo, —Br), and iodine (iodo, —I). For example, “haloalkyl” means an alkyl substituent wherein at least one hydrogen atom is replaced with a halogen atom and “haloalkoxy” means an alkoxy substituent wherein at least one hydrogen atom is replaced with a halogen atom.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR+ (as in N-substituted pyrrolidinyl).

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-6 carbon atoms. In some embodiments, aliphatic groups contain 1-4 carbon atoms, and in yet other embodiments aliphatic groups contain 1-3 carbon atoms. Aliphatic groups include, but are not limited to, alkyl, alkenyl, alkynyl, carbocycle. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein, refers to a saturated, straight- (linear) or branched-chain hydrocarbon radical typically containing from 1 to 20 carbon atoms. For example, “C₁-C₆ alkyl” contains from one to six carbon atoms. Examples of alkyl radicals include, but are not limited to, methyl, ethyl, propyl, isopropyl, n-butyl, sec-butyl, tent-butyl, neopentyl, n-hexyl, heptyl, octyl radicals and the like.

The term “alkenyl” as used herein, denotes a straight- or branched-chain hydrocarbon radical containing one or more double bonds and typically from 2 to 20 carbon atoms. For example, “C₂-C₈ alkenyl” contains from two to eight carbon atoms. Alkenyl groups include, but are not limited to, for example, ethenyl, propenyl, butenyl, 1-methyl-2-buten-1-yl, heptenyl, octenyl and the like.

The term “alkoxy” as used herein, refers to group —Oalkyl, wherein alkyl is as defined herein. For example, the alkoxy group may be —O(C₁-C₆ alkyl). Examples of alkoxy groups include, without being limited to, —OMe, —OEt, —OⁱPr etc.

The terms “cycloalkyl”, “alicyclic”, “carbocyclic” and equivalent expressions refer to a group comprising a saturated or partially unsaturated (non-aromatic) carbocyclic ring in a monocyclic or polycyclic ring system, including spiro (sharing one atom), fused (sharing at least one bond) or bridged (sharing two or more bonds) carbocyclic ring systems, having from three to fifteen ring members. Partially unsaturated carbocyclic rings can also be referred to as “cycloalkenyl”. Examples of cycloalkyl or cycloalkenyl groups can include, without limitation, cyclopropyl, cyclobutyl, cyclopentyl, cyclopenten-1-yl, cyclopenten-2-yl, cyclopenten-3-yl, cyclohexyl, cyclohexen-1-yl, cyclohexen-2-yl, cyclohexen-3-yl, cycloheptyl, bicyclo[4,3,0]nonanyl, norbornyl, and the like. The term “cycloalkyl” includes both unsubstituted cycloalkyl groups and substituted cycloalkyl groups. The term “C₃-Cₙcycloalkyl” refers to a cycloalkyl group having from 3 to the indicated “n” number of carbon atoms in the ring structure.

As used herein, the terms “heterocycle”, “heterocycloalkyl”, “heterocyclyl”, “heterocyclic radical” and “heterocyclic ring” are used interchangeably and refer to a chemically stable 3- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 1-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl). A heterocyclic ring can be attached to its pendant group at any heteroatom or carbon atom that results in a chemically stable structure and any of the ring atoms can be optionally substituted. Examples of heterocycloalkyl groups include, but are not limited to, 1,3-dioxolanyl, pyrrolidinyl, pyrrolidonyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, tetrahydrofuranyl, tetrahydropyranyl, tetrahydrothiopyranyl, tetrahydrodithienyl, tetrahydrothienyl, thiomorpholino, thioxanyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, 2H-pyranyl, 4H-pyranyl, dioxanyl, dithianyl, dithiolanyl, dihydropyranyl, dihydrothienyl, dihydrofuranyl, 3-azabicyclo[3,1,0]hexanyl, 3-azabicyclo[4,1,0]heptanyl, quinolizinyl, quinuclidinyl, tetrahydroquinolinyl, tetrahydroisoquinolinyl, decahydroquinolinyl, and the like. Heterocyclic groups also include groups in which a heterocyclic ring is fused to one or more aryl, heteroaryl, or cycloaliphatic rings, such as indolinyl, 3H-indolyl, chromanyl, chromenyl, phenanthridinyl, 2-azabicyclo[2.2.1]heptanyl, octahydroindolyl, or tetrahydroquinolinyl, where the radical or point of attachment is on the heterocyclyl ring. A heterocyclyl group may be mono- or bicyclic. Another example includes a group

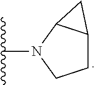 [SMILES: *N1CCC2CC21]

As used herein, the term “partially unsaturated” refers to a ring moiety that includes at least one double or triple bond between ring atoms but is not aromatic. The term “partially unsaturated” is intended to encompass rings having multiple sites of unsaturation, but is not intended to include aryl or heteroaryl moieties, as herein defined.

The term “aryl” used herein refers to a monocyclic moiety or to a bicyclic or tricyclic fused ring system having a total of six to 15 ring members, wherein at least one ring in the system is aromatic and wherein each ring in the system contains three to seven ring members. The term “aryl” may be used interchangeably with the term “aryl ring”. In certain embodiments of the present description, “aryl” refers to an aromatic ring system which includes, but not limited to, phenyl, biphenyl, naphthyl, azulenyl, anthracyl and the like, which may bear one or more substituents.

The term “heteroaryl” used alone or as part of a larger moiety, refers to groups having 5 to 18 ring atoms, preferably 5, 6, or 9 ring atoms; having 6, 10, or 14 π electrons shared in a cyclic array; and having, in addition to carbon atoms, from one to five heteroatoms. The term “heteroatom” includes but is not limited to nitrogen, oxygen, or sulfur, and includes any oxidized form of nitrogen or sulfur, and any quaternized form of a basic nitrogen. A heteroaryl may be a single ring, or two or more fused rings. Heteroaryl groups include, without limitation, thienyl, furanyl (furyl), thienyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, triazinyl, furopyridinyl, indolizinyl, purinyl, naphthyridinyl, and pteridinyl. The term “heteroaryl” as used herein, also includes groups in which a heteroaromatic ring is fused to one or more aryl, cycloaliphatic, or heterocyclyl rings, where the radical or point of attachment is on the heteroaromatic ring. Non-limiting examples include indolyl, 3H-indolyl, isoindolyl, benzothienyl (benzothiophenyl), benzofuranyl, dibenzofuranyl, indazolyl, benzimidazolyl, benzoxazolyl, benzothiazolyl, quinolyl (quinolinyl), isoquinolyl (isoquinolinyl), quinolonyl, isoquinolonyl, cinnolinyl, phthalazinyl, quinazolinyl, quinoxalinyl, 4H-quinolizinyl, carbazolyl, acridinyl, phenanthridinyl, phenazinyl, phenothiazinyl, phenoxazinyl, tetrahydroquinolinyl, tetrahydroisoquinolinyl, and pyrido[2,3-b]-l,4-oxazin-3(4H)-one. A heteroaryl group can be attached to its pendant group at any heteroatom or carbon atom that results in a chemically stable structure. A heteroaryl group may be mono- or bicyclic. Heteroaryl groups include rings that are optionally substituted.

As described herein, various chemical groups present in the compounds of the present description can be optionally substituted. In general, the term “substituted” means that one or more hydrogens of the designated moiety are replaced with a suitable substituent. Unless otherwise indicated, a substituted group may have a suitable substituent at each substitutable position of the group, and when more than one position in any given structure may be substituted with more than one substituent selected from a specified group, the substituent may be either the same or different at each position. Combinations of substituents envisioned under the present description are preferably those that result in the formation of chemically stable or chemically feasible compounds. The term “chemically stable”, as used herein, refers to compounds that are not substantially altered when subjected to conditions to allow for their production, detection, and, in certain embodiments, their recovery, purification, and use for one or more of the purposes disclosed herein.

When a group is substituted, it can be substituted by independent replacement of one, two, or three or more of the hydrogen atoms with substituents including, but not limited to halogen (i.e., F, Cl, Br, I), OH, CO₂H, alkoxy such as methoxy, protected alkoxy, alkyl such as methyl, oxo, thiooxo, NO₂, CN, CF₃, NH₂, protected amino, —CH₂OH, —CF₃, —CHF₂, —CH₂F, —SO₂NH(CH₂)₃OH and pyrazolyl.

The expression “pharmaceutically acceptable salt” refers to those salts of the compounds formed by the process of the present description which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the present description, or separately by reacting a free base function of the compound with a suitable organic or inorganic acid (acid addition salts) or by reacting an acidic function of the compound with a suitable organic or inorganic base (base-addition salts). Examples of pharmaceutically acceptable salts include, but are not limited to, nontoxic acid addition salts, or salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, cam phorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative base addition alkali or alkaline earth metal salts include sodium, lithium, potassium, calcium, or magnesium salts, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, sulfonate and aryl sulfonate.

The term “solvate” refers to a physical association of one of the present compounds with one or more solvent molecules. This physical association includes hydrogen bonding. In certain instances, the solvate will be capable of isolation, for example when one or more solvent molecules are incorporated in the crystal lattice of a crystalline solid. “Solvate” encompasses both solution-phase and isolable solvates. Exemplary solvates include, without limitation, hydrates, hemihydrates, ethanolates, hemiethanolates, n-propanolates, iso-propanolates, 1-butanolates, 2-butanolate, and solvates of other physiologically acceptable solvents. The compounds as herein described also include each of their solvates and mixtures thereof.

As used herein, the term “pharmaceutically acceptable ester” refers to esters of the compounds formed by the process of the present description which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The expression “pharmaceutically acceptable prodrugs” as used herein refers to those prodrugs of the compounds formed by the process of the present description which are, within the scope of sound medical judgment, suitable for use in contact with the tissues of humans and lower animals with undue toxicity, irritation, allergic response, and the like, commensurate with a reasonable benefit/risk ratio, and effective for their intended use. “Prodrug”, as used herein means a compound which is convertible in vivo by metabolic means (e.g. by hydrolysis) to afford any compound delineated by the formulae of the instant description. Various forms of prodrugs are known in the art.

Combinations of substituents and variables envisioned by the present description are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

### Compounds

The compounds of the present application may be prepared by conventional chemical synthesis, such as exemplified in the general scheme provided hereafter and in Examples 1A and 1B for instance. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired products of the present description. Synthetic chemistry transformations and/or protecting group methodologies (protection and deprotection) useful in synthesizing the compounds described herein are known in the art. The synthesized compounds can be separated from a reaction mixture and further purified by standard methods such as column chromatography, high pressure liquid chromatography, or recrystallization.

The compounds of the present description may be modified by appending various functionalities via any synthetic means delineated herein to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof. As such, the following embodiments are present alone or in combination if applicable.

The present application provides compounds of general Formula (I), as well as their pharmaceutically acceptable salts, solvates, esters or prodrugs thereof:

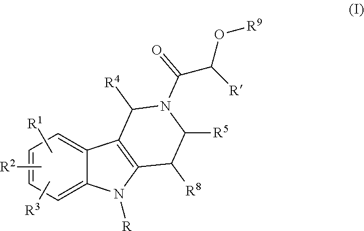

wherein:
R¹, R² and R³ independently represent hydrogen, halogen, CN, unsubstituted or substituted C₁-C₆ alkoxy, unsubstituted or substituted C₂-C₆ alkenyl, unsubstituted or substituted linear or branched C₁-C₆ alkyl, unsubstituted or substituted C₃-C₆ cycloalkyl or cycloalkenyl, unsubstituted or substituted C₆ aryl, unsubstituted or substituted 4 to 10 membered heterocycloalkyl being saturated or partially unsaturated, unsubstituted or substituted 4 to 10 membered heteroaryl, —NR¹⁰C(═O)R¹¹, —C(═O)NR¹²R¹³ or —CHR¹⁴R¹⁵,
   with at least one of R¹, R² and R³ being different than hydrogen;
R⁴, R⁵ and R⁸ independently represent H, unsubstituted or substituted linear or branched C₁-C₆ alkyl, —CH₂Ph, with at least one of R⁴ and R⁵ being different than hydrogen; or
   R⁵ and R⁸ are linked together to form a C₅-C₆ cycloalkyl;
R and R′ independently represent H or linear or branched C₁-C₃alkyl;
R⁹ is H or linear or branched C₁-C₃alkyl;
R¹⁰ is H or linear or branched C₁-C₃alkyl;
R¹¹ is unsubstituted or substituted linear or branched C₁-C₃alkyl;
R¹² is H or linear or branched C₁-C₃alkyl;
R¹³ is linear or branched C₁-C₃alkyl;
R¹⁴ is H or linear or branched C₁-C₃alkyl;
R¹⁵ is unsubstituted or substituted C₆ aryl, unsubstituted or substituted C₃-C₆ cycloalkyl or unsubstituted or substituted 5 to 6 membered heterocycloalkyl.

In some embodiments, the heterocycloalkyl and heteroaryl groups include 1 to 3 heteroatoms independently selected from the group consisting of N, O and S.

In other embodiments, when any alkyl, alkoxy, alkenyl, cycloalkyl, cycloalkenyl, saturated or partially unsaturated heterocycloalkyl, aryl or heteroaryl groups are substituted, these groups are independently substituted with 1 to 3 substituents selected from the group consisting of halogen, hydroxy, methoxy, methyl, oxo (═O), CN, —NH₂, —NH(C₁-C₃alkyl), —N(C₁-C₃alkyl)₂, —NH(CO)CF₃, —CH₂OH, —CF₃, —CHF₂, —CH₂F, —SO₂NH(CH₂)₃OH and pyrazolyl.

In some embodiments, there is provided a compound of formula (I):

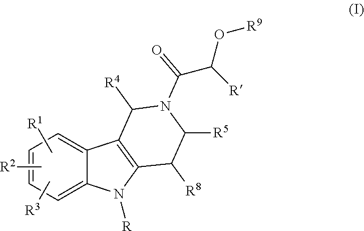

or a pharmaceutically acceptable salt, solvate, ester or prodrug thereof,
wherein:
R¹, R² and R³ independently represent hydrogen, halogen, CN, unsubstituted or substituted C₁-C₆ alkoxy, unsubstituted or substituted C₂-C₆ alkenyl, unsubstituted or substituted linear or branched C₁-C₆ alkyl, unsubstituted or substituted C₃-C₆ cycloalkyl or cycloalkenyl, unsubstituted or substituted C₆ aryl, unsubstituted or substituted 4 to 10 membered heterocycloalkyl being saturated or partially unsaturated, unsubstituted or substituted 4 to 10 membered heteroaryl, —NR¹⁰C(═O)R¹¹, —C(═O)NR¹²R¹³ or —CHR¹⁴R¹⁵,
   with at least one of R¹, R² and R³ being different than hydrogen;
R⁴, R⁵ and R⁸ independently represent H, unsubstituted or substituted linear or branched C₁-C₆ alkyl, —CH₂Ph, with at least one of R⁴ and R⁵ being different than hydrogen; or
   R⁵ and R⁸ are linked together to form a C₅-C₆ cycloalkyl;
R and R′ independently represent H or linear or branched C₁-C₃alkyl;
R⁹ is H or linear or branched C₁-C₃alkyl;
R¹⁰ is H or linear or branched C₁-C₃alkyl;
R¹¹ is unsubstituted or substituted linear or branched C₁-C₃alkyl;
R¹² is H or linear or branched C₁-C₃alkyl;
R¹³ is linear or branched C₁-C₃alkyl;
R¹⁴ is H or linear or branched C₁-C₃alkyl,
R¹⁵ is unsubstituted or substituted C₆ aryl, unsubstituted or substituted C₃-C₆ cycloalkyl or unsubstituted or substituted 5 to 6 membered heterocycloalkyl;
   wherein the heterocycloalkyl and heteroaryl groups include 1 to 3 heteroatoms independently selected from the group consisting of N, O and S;
   wherein, when any alkyl, alkoxy, alkenyl, cycloalkyl, cycloalkenyl, saturated or partially unsaturated heterocycloalkyl, aryl or heteroaryl groups are substituted, these groups are independently substituted with 1 to 3 substituents selected from the group consisting of halogen, hydroxy, methoxy, methyl, oxo (═O), CN, —NH₂, —NH(C₁-C₃alkyl), —N(C₁-C₃alkyl)₂, —NH(CO)CF₃, —CH₂OH, —CF₃, —CHF₂, —CH₂F, —SO₂NH(CH₂)₃OH and pyrazolyl.

In some embodiments, the compound of formula (I) or the pharmaceutically acceptable salt, solvate, ester or prodrug thereof, can be a compound of formula (Ia) or (Ib)

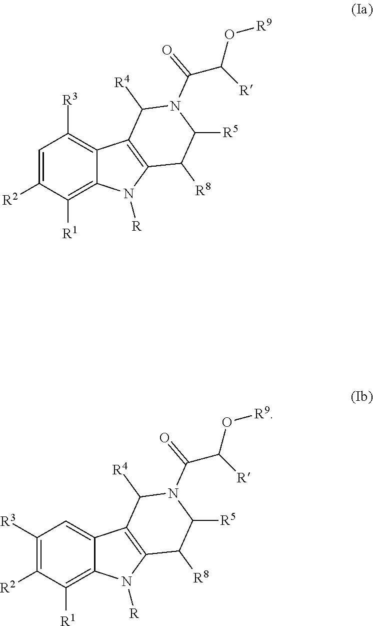

In some embodiments, the compounds is a compound of formula (Ia) or a pharmaceutically acceptable salt, solvate, ester or prodrug thereof.

In some embodiments, the groups R¹ and R² can be both different than hydrogen.

In some embodiments, the groups R¹ and R² independently can represent halogen, CN, unsubstituted or substituted C₁-C₆ alkoxy, unsubstituted or substituted C₂-C₆ alkenyl, or unsubstituted or substituted linear or branched C₁-C₆ alkyl, wherein, when any alkyl, alkoxy, or alkenyl groups are substituted, these groups are independently substituted with 1 to 3 halogen atoms.

In some embodiments, the groups R¹ and R² independently can represent halogen, CN, unsubstituted C₁-C₂ alkoxy, C₁-C₂ alkoxy substituted with 1 to 3 halogen atoms, unsubstituted C₁-C₂ alkyl, C₁-C₂ alkyl substituted with 1 to 3 halogen atoms, or unsubstituted C₂-C₃ alkenyl.

In some embodiments, the groups R¹ and R² independently can represent halogen or methyl.

In some embodiments, the groups R¹ and R² can both represent halogen.

In some embodiments, the groups R³ can represent hydrogen, halogen, CN, —NR¹⁰C(═O)R¹¹, or —C(═O)NR¹²R¹³ with R¹⁰ and R¹² representing hydrogen and R¹¹ and R¹³ representing —CH³, unsubstituted C₁-C₃ alkoxy, unsubstituted C₂-C₃ alkenyl, unsubstituted C₁-C₃ alkyl, or C₁-C₃ alkyl substituted with 1 to 3 groups selected from halogen, methoxy, CN, —NH₂, —NH(C₁-C₃alkyl), and —N(C₁-C₃alkyl)₂.

In some embodiments, R³ can represent

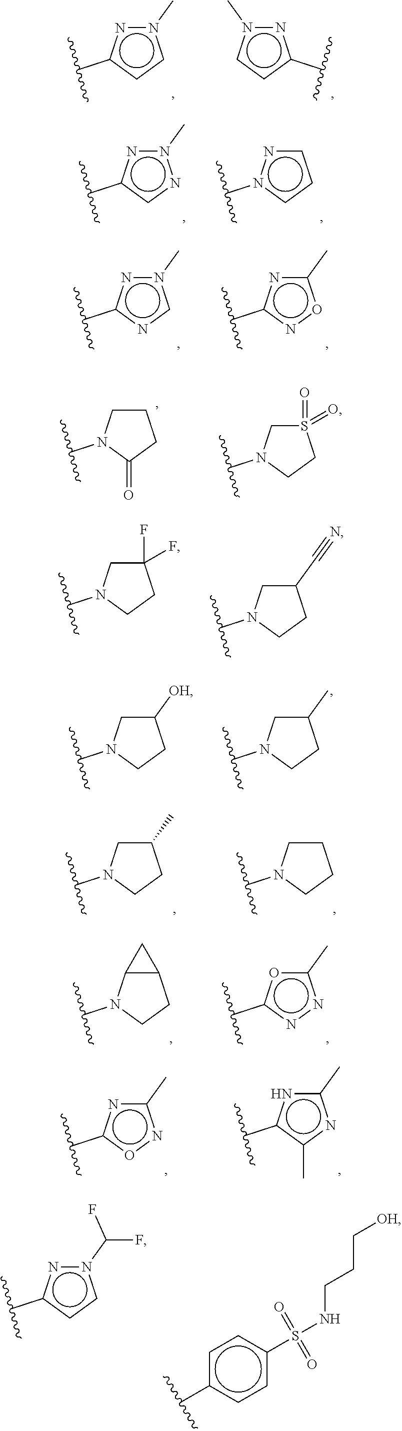

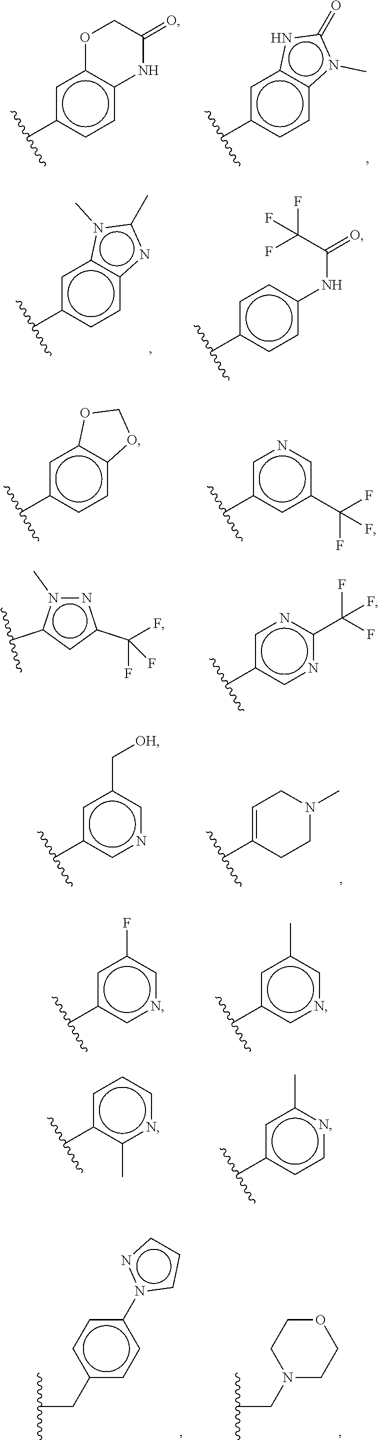

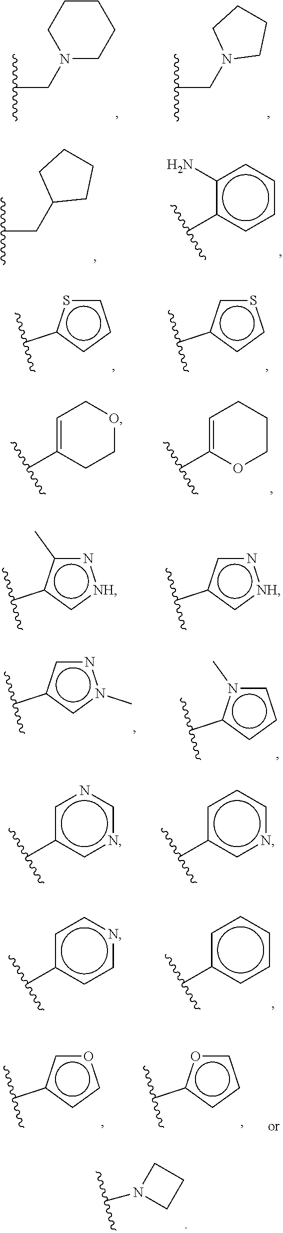

In some other embodiments, R³ can represent hydrogen, halogen, CN, —CH₃, —OCH₃, —CH═CH₂, —CH₂CH₂OCH₃, —CH₂N(CH)₂, —NR¹⁰C(═O)R¹¹, or —C(═O)NR¹²R¹³ with R¹⁰ and R¹² represent hydrogen and R¹¹ and R¹³ represent —CH₃,

or R³ can represent

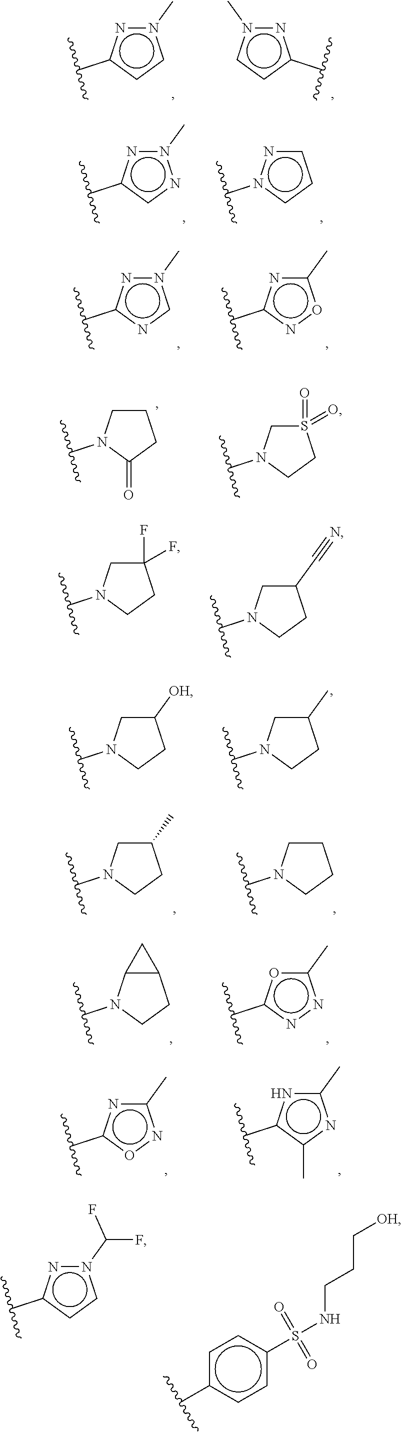

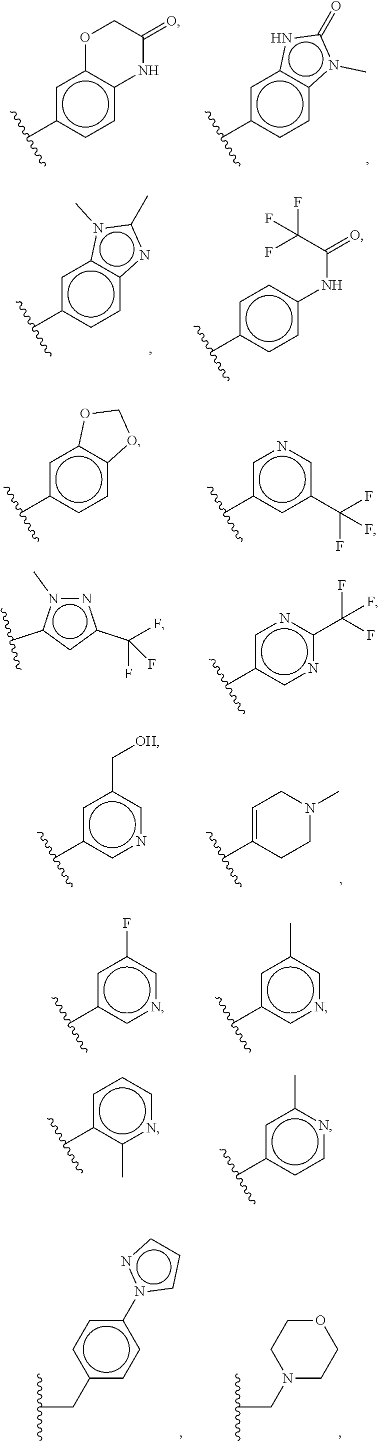

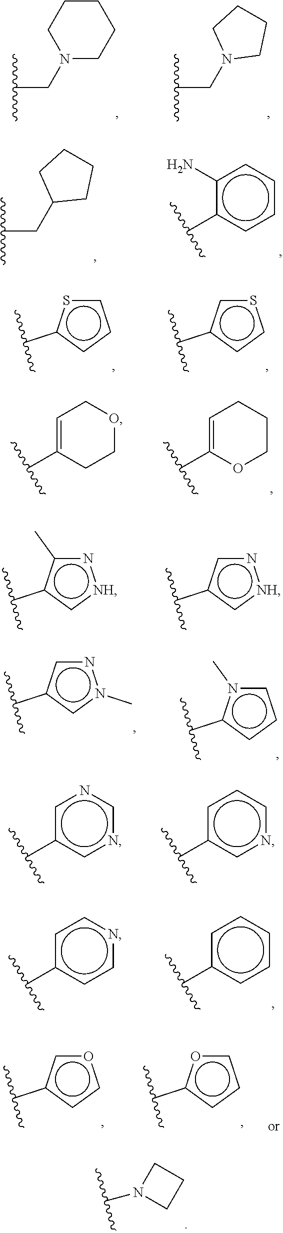

In certain embodiments, R³ represents hydrogen or

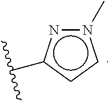

In further embodiments, R¹ and R² represent halogen or methyl and R³ represents hydrogen.

In some embodiments, the group R can be hydrogen. In certain embodiments, the group R′ can represent hydrogen.

In some embodiments, the groups R⁴, R⁵ and R⁸ independently can represent H, unsubstituted or substituted linear or branched C₁-C₃ alkyl, or —CH₂Ph, with at least one of R⁴ and R⁵ being different than hydrogen; or R⁵ and R⁸ are linked together to form a C₅-C₆ cycloalkyl; and when the C₁-C₃ alkyl is substituted, the substituent(s) is(are) halogen.

In some embodiments, the groups R⁴, R⁵ and R⁸ independently can represent H, —CH₃, —CH₂CH₃, —CH(CH₃)₂, —CF₃, or —CH₂Ph, with at least one of R⁴ and R⁵ being different than hydrogen; or R⁵ and R⁸ are linked together to form a C₆ cycloalkyl and R⁴ represents hydrogen.

In some embodiments, at least one of R⁴ and R⁵ is —CH₃.

In some embodiments, the group R⁹ can represents hydrogen or methyl. In some embodiments, R⁹ represents hydrogen. In other embodiments, R⁹ represents methyl.

In further embodiments, the compound of the present disclosure can be a compound of formula (Ic), or a pharmaceutically acceptable salt, solvate, ester or prodrug thereof,

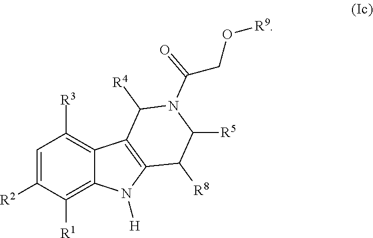

In the formula (Ic), the groups R¹, R², R³, R⁴, R⁵, R⁸, and R⁹, can have anyone of the definitions mentioned above with respect to formula (I), (Ia) and or (Ib).

In certain embodiments, the compound can be a compound of formula (Ic) where the groups R¹ and R² independently can represent halogen or methyl and R³ represents hydrogen.

In some embodiments, the compound can be a compound of formula (Ic) where the group R¹ can represent halogen, R² can represent halogen or methyl, and R³ can represent hydrogen.

In some embodiments, the compound can be a compound of formula (Ic) where the groups R⁴, R⁵ and R⁸ independently can represent H, —CH₃, —CH₂CH₃, —CH(CH₃)₂, —CF₃, or —CH₂Ph, with at least one of R⁴ and R⁵ being different than hydrogen; or with R⁵ and R⁸ being linked together to form a C₆ cycloalkyl and R⁴ representing hydrogen.

In some embodiments, the compound can be a compound of formula (Ic) where R⁹ can represent hydrogen or methyl.

In some embodiments, the compound can be a compound of formula (Ic) where R⁹ can represent methyl.

In a further embodiment, this application relates to a compound selected from Compounds 1 to 140 as defined in Table 1 below, or a pharmaceutically acceptable salt, solvate, or prodrug thereof. In yet another embodiment, this application relates to a compound selected from Compounds 102 to 126 as defined in Table 1 below, or a pharmaceutically acceptable salt, solvate, or prodrug thereof. Table 1 also includes the reference to the synthesis method that can be used to prepare the compounds, corresponding to either Example 1A or Example 1B included in the Examples section below.

TABLE 1
Compounds 1 to 140

| | | Synthesis |
|---|---|---|
| Compound | | performed according |
| Number | Compound structure | to Example # |
| 1 | 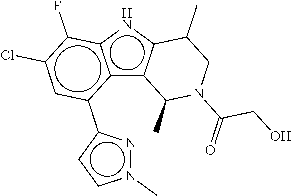 | 1B |
| 2 | 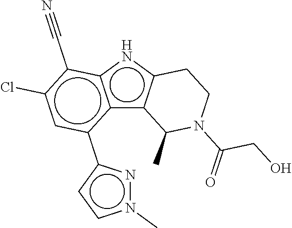 | 1B |
| 3 | 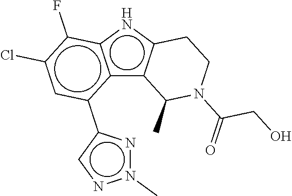 | 1B |
| 4 | 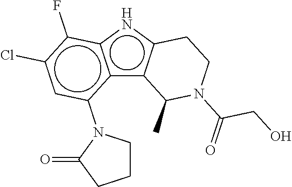 | 1B |
| 5 | 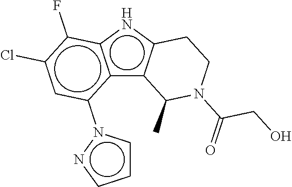 | 1B |
| 6 | 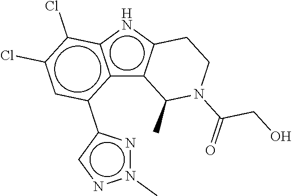 | 1B |
| 7 | 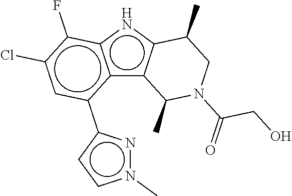 | 1B |
| 8 | 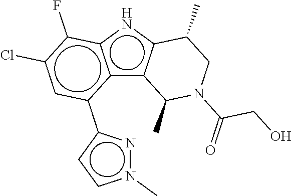 | 1B |
| 9 | 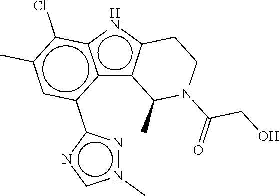 | 1B |
| 10 | 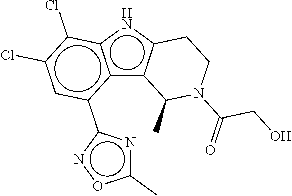 | 1B |
| 11 | 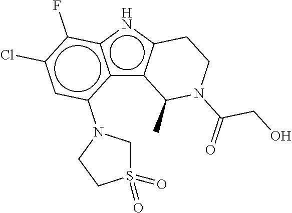 | 1B |
| 12 | 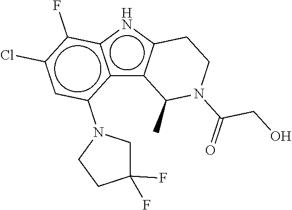 | 1B |
| 13 | 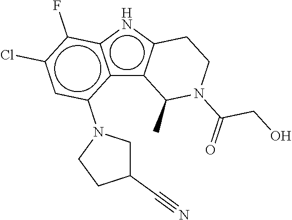 | 1B |
| 14 | 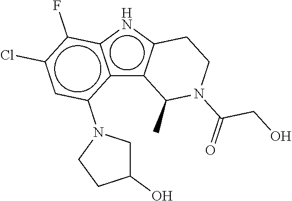 | 1B |
| 15 | 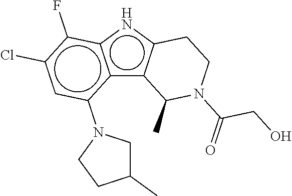 | 1B |
| 16 | 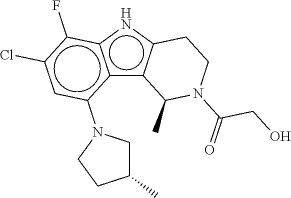 | 1B |
| 17 | 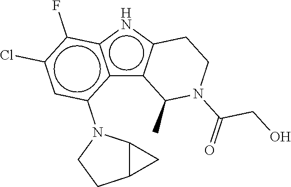 | 1B |
| 18 | 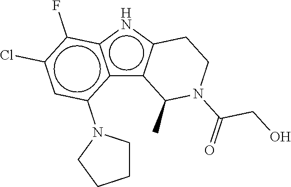 | 1B |
| 19 | 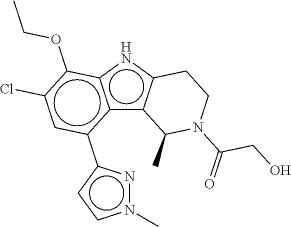 | 1B |
| 20 | 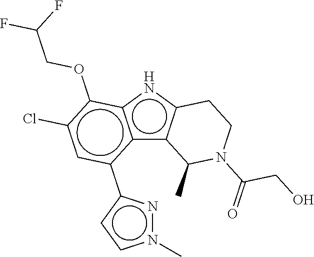 | 1B |
| 21 | 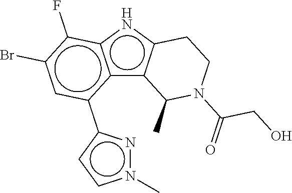 | 1B |
| 22 | 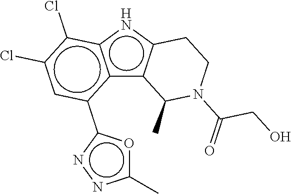 | 1B |
| 23 | 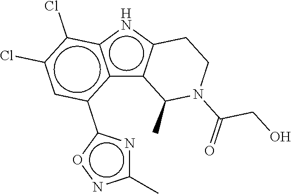 | 1B |
| 24 | 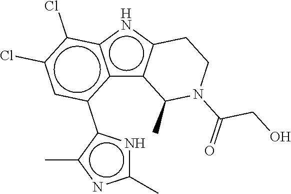 | 1B |
| 25 | 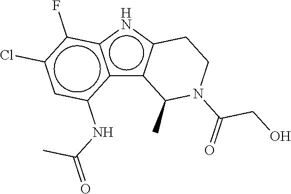 | 1B |
| 26 | 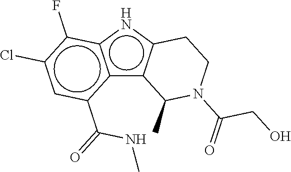 | 1B |
| 27 | 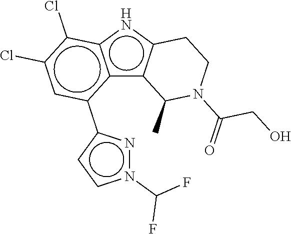 | 1B |
| 28 | 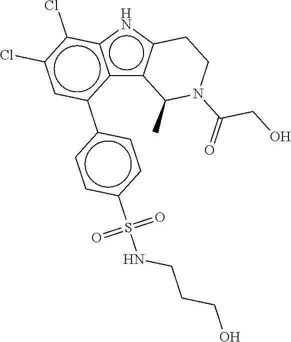 | 1B |
| 29 | 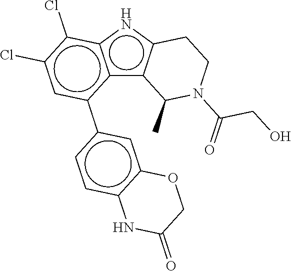 | 1B |
| 30 | 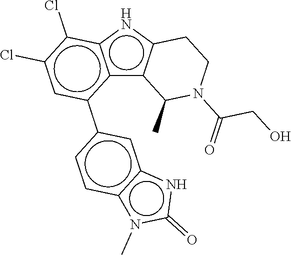 | 1B |
| 31 | 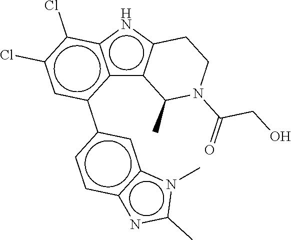 | 1B |
| 32 | 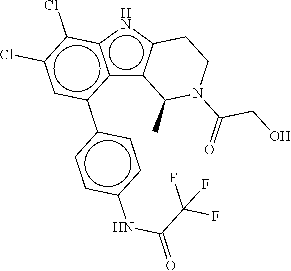 | 1B |
| 33 | 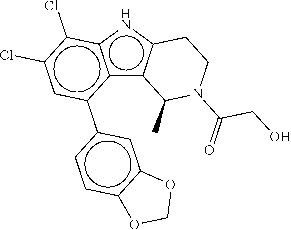 | 1B |
| 34 | 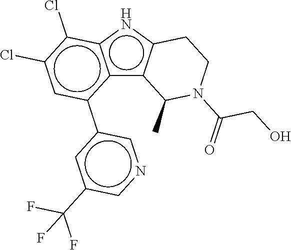 | 1B |
| 35 | 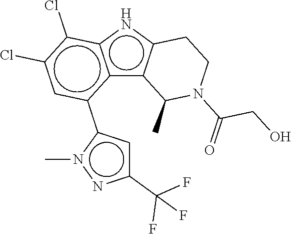 | 1B |
| 36 | 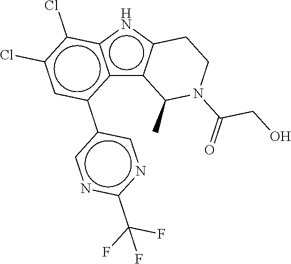 | 1B |
| 37 | 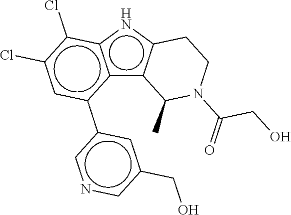 | 1B |
| 38 | 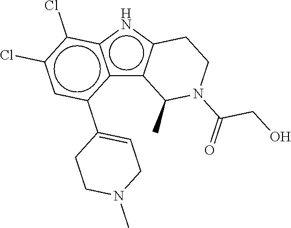 | 1B |
| 39 | 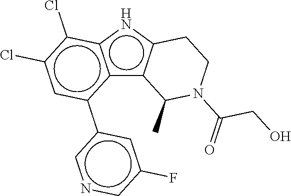 | 1B |
| 40 | 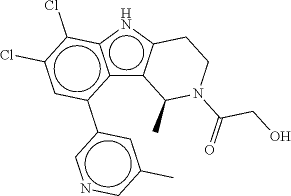 | 1B |
| 41 | 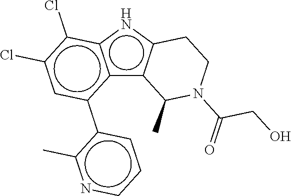 | 1B |
| 42 | 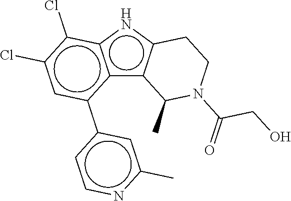 | 1B |
| 43 | 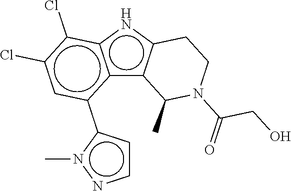 | 1B |
| 44 | 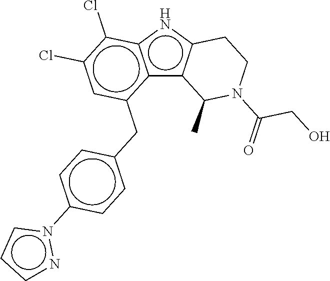 | 1B |
| 45 | 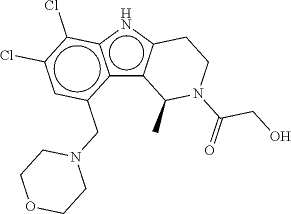 | 1B |
| 46 | 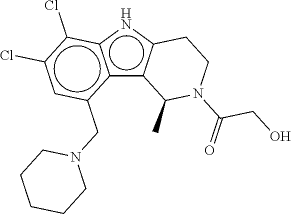 | 1B |
| 47 | 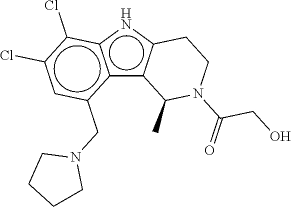 | 1B |
| 48 | 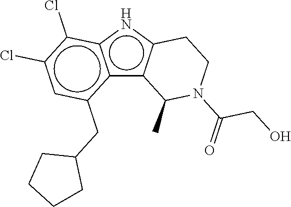 | 1B |
| 49 | 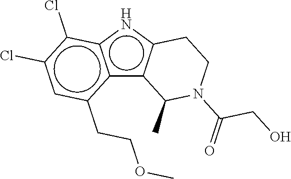 | 1B |
| 50 | 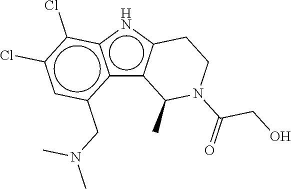 | 1B |
| 51 | 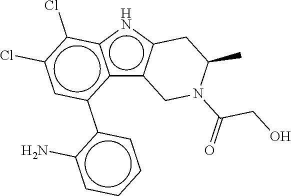 | 1B |
| 52 | 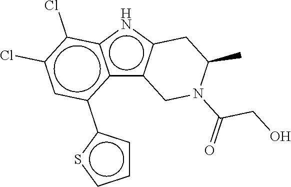 | 1B |
| 53 | 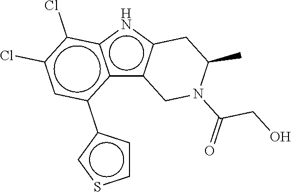 | 1B |
| 54 | 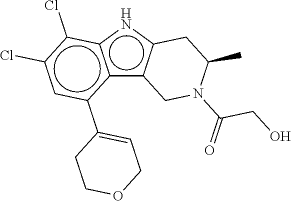 | 1B |
| 55 | 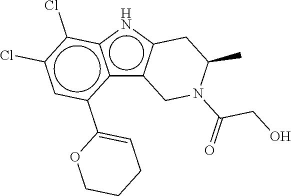 | 1B |
| 56 | 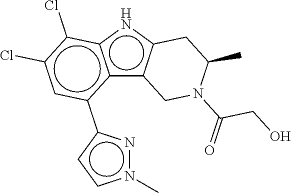 | 1B |
| 57 | 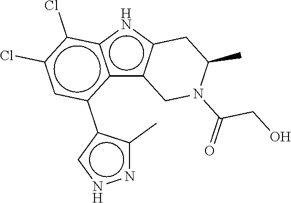 | 1B |
| 58 | 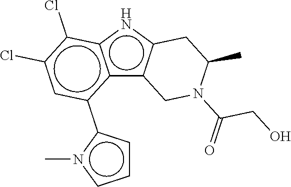 | 1B |
| 59 | 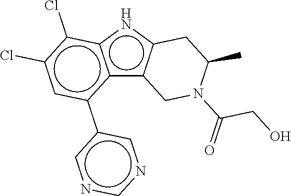 | 1B |
| 60 | 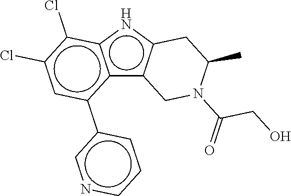 | 1B |
| 61 | 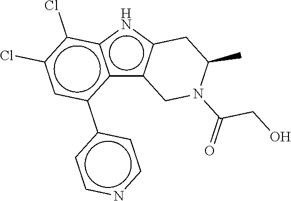 | 1B |
| 62 | 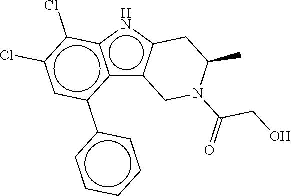 | 1B |
| 63 | 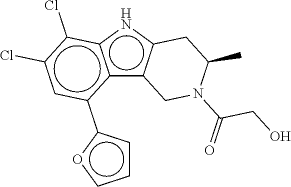 | 1B |
| 64 | 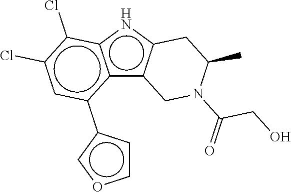 | 1B |
| 65 | 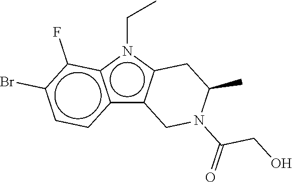 | 1A |
| 66 | 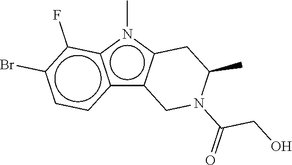 | 1A |
| 67 | 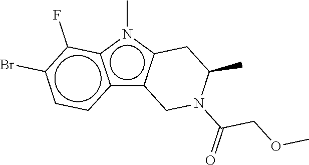 | 1A |
| 68 | 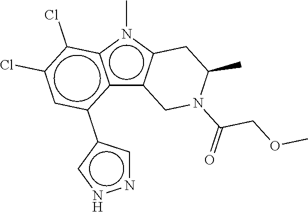 | 1B |
| 69 | 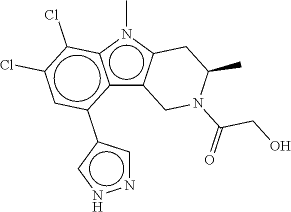 | 1B |
| 70 | 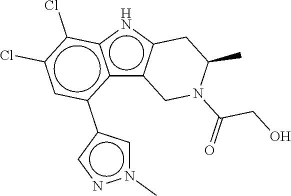 | 1B |
| 71 | 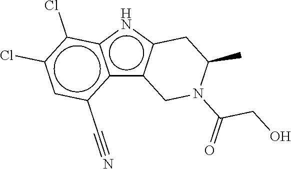 | 1B |
| 72 | 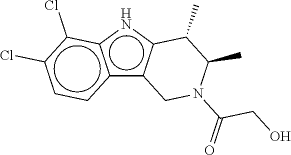 | 1A |
| 73 | 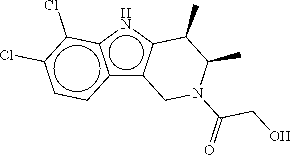 | 1A |
| 74 | 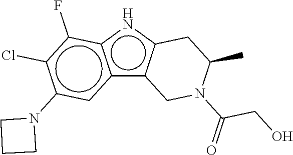 | 1A |
| 75 | 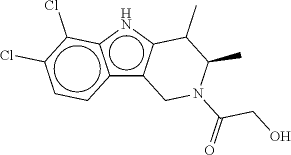 | 1A |
| 76 | 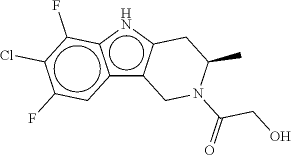 | 1A |
| 77 | 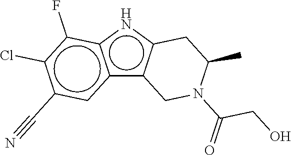 | 1A |
| 78 | 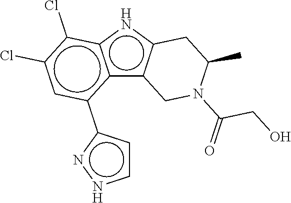 | 1B |
| 79 | 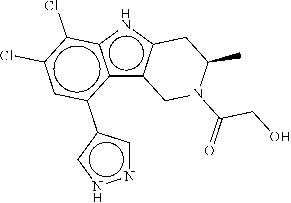 | 1B |
| 80 | 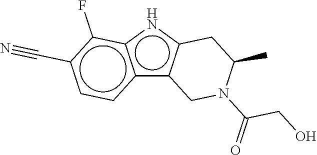 | 1A |
| 81 | 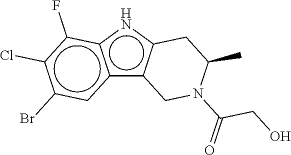 | 1A |
| 82 | 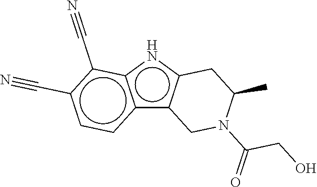 | 1A |
| 83 | 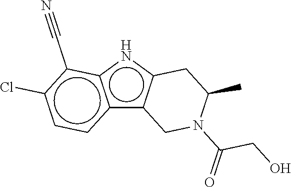 | 1A |
| 84 | 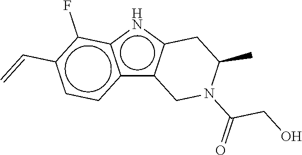 | 1A |
| 85 | 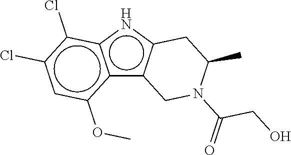 | 1A |
| 86 | 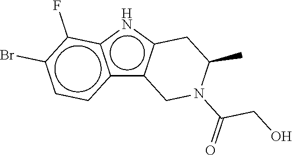 | 1A |
| 87 | 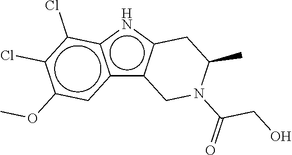 | 1A |
| 88 | 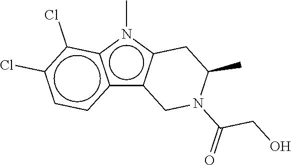 | 1A |
| 89 | 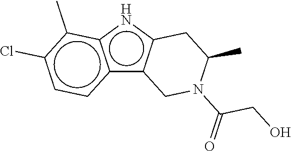 | 1A |
| 90 | 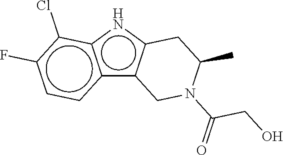 | 1A |
| 91 | 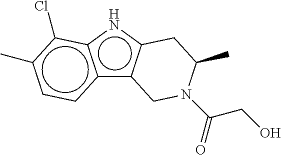 | 1A |
| 92 | 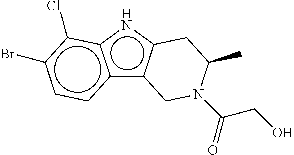 | 1A |
| 93 | 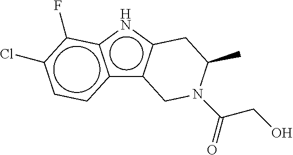 | 1A |
| 94 | 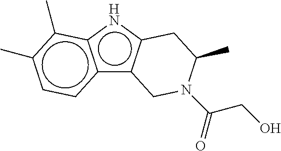 | 1A |
| 95 | 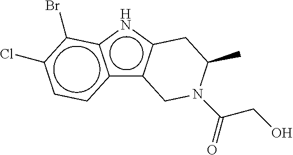 | 1A |
| 96 | 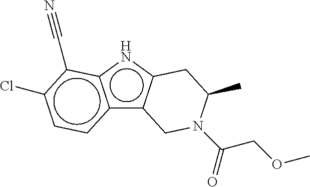 | 1A |
| 97 | 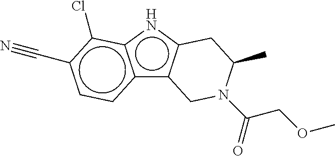 | 1A |
| 98 | 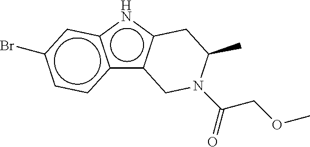 | 1A |
| 99 | 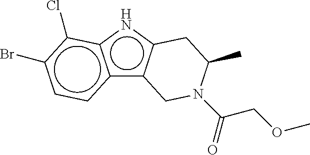 | 1A |
| 100 | 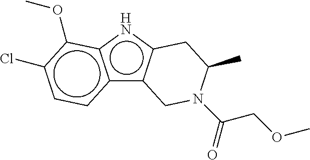 | 1A |
| 101 | 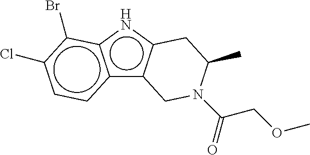 | 1A |
| 102 | 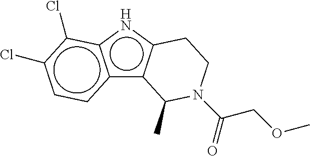 | 1A |
| 103 | 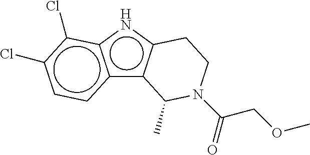 | 1A |
| 104 | 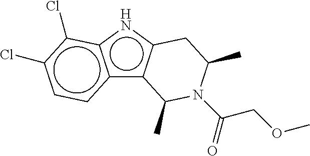 | 1A |
| 105 | 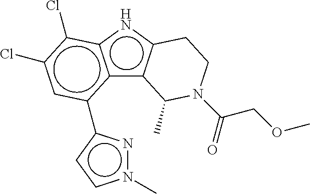 | 1A |
| 106 | 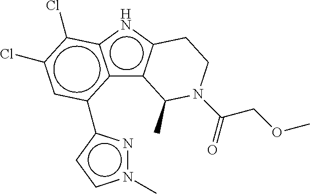 | 1A |
| 107 | 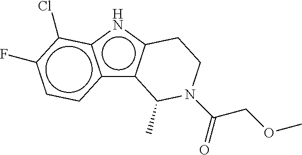 | 1A |
| 108 | 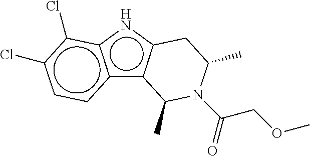 | 1A |
| 109 | 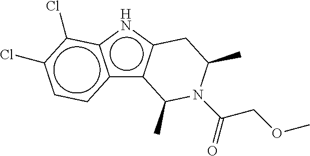 | 1A |
| 110 | 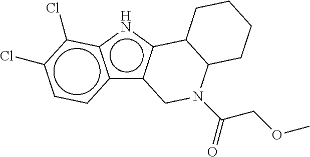 | 1A |
| 111 | 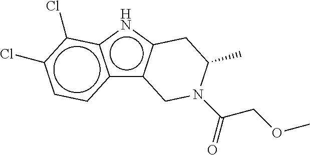 | 1A |
| 112 | 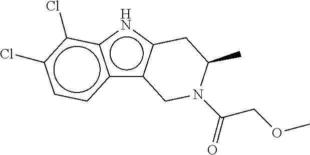 | 1A |
| 113 | 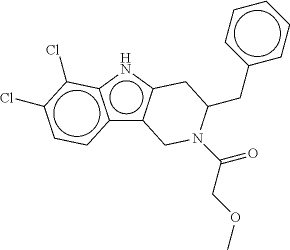 | 1A |
| 114 | 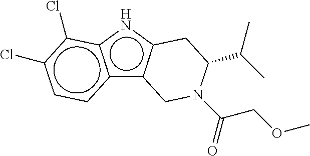 | 1A |
| 115 | 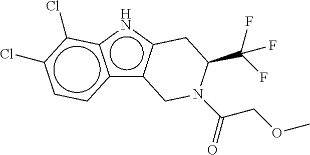 | 1A |
| 116 | 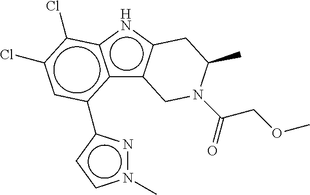 | 1B |
| 117 | 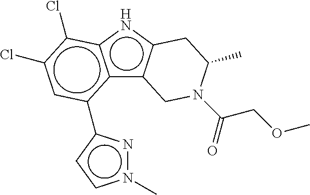 | 1B |
| 118 | 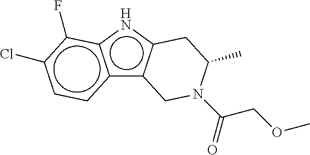 | 1A |
| 119 | 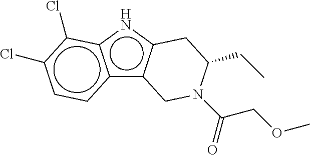 | 1A |
| 120 | 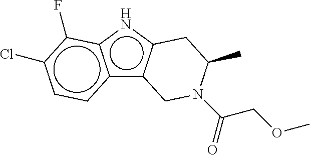 | 1A |
| 121 | 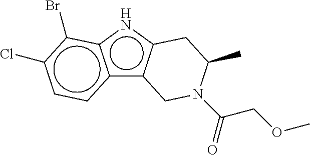 | 1A |
| 122 | 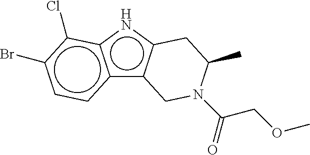 | 1A |
| 123 | 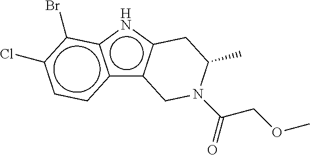 | 1A |
| 124 | 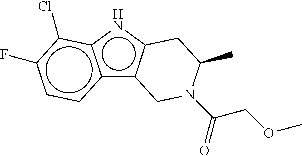 | 1A |
| 125 | 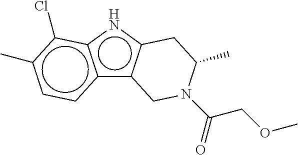 | 1A |
| 126 | 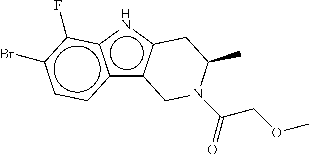 | 1A |
| 127 | 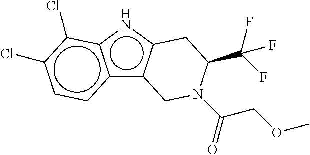 | 1A |
| 128 | 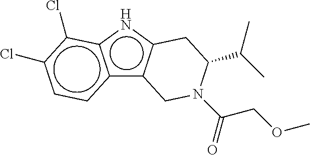 | 1A |
| 129 | 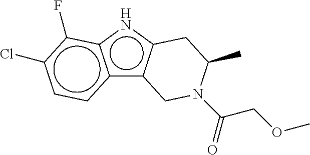 | 1A |
| 130 | 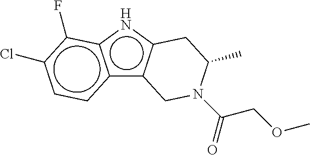 | 1A |
| 131 | 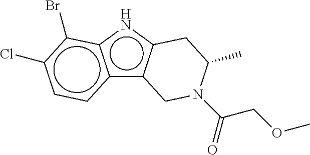 | 1A |
| 132 | 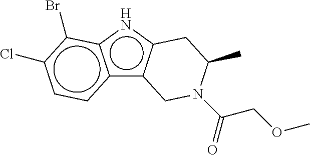 | 1A |
| 133 | 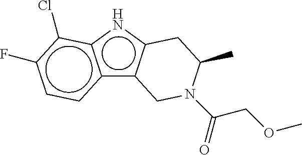 | 1A |
| 134 | 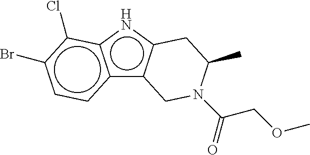 | 1A |
| 135 | 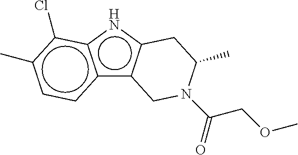 | 1A |
| 136 | 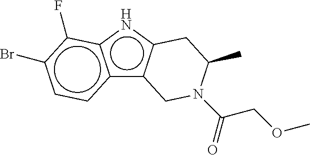 | 1A |
| 137 | 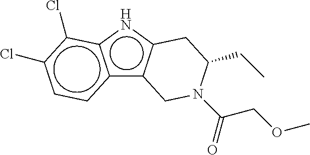 | 1A |
| 138 | 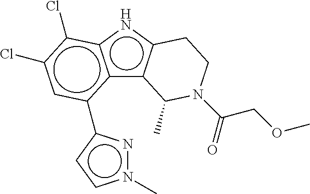 | 1B |
| 139 | 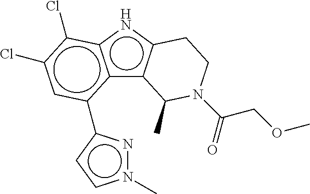 | 1B |
| 140 | 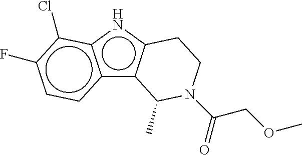 | 1A |

### Methods, Uses, Formulations and Administration

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in treatment, healing, prevention, or amelioration of a disease, disorder, or side effect, or a decrease in the rate of advancement of a disease or disorder. The term also includes within its scope amounts effective to enhance normal physiological function.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the term “cGAS inhibitor” denotes a compound which inhibits the cyclic GMP-AMP synthase.

The term “patient or subject” as used herein refers to a mammal. A subject therefore refers to, for example, dogs, cats, horses, cows, pigs, guinea pigs, and the like. Preferably the subject is a human. When the subject is a human, the subject may be either a patient or a healthy human.

In some embodiments, the disease or condition can be autoinflammation or an autoimmune disease, such as systemic lupus erythematosus (SLE), Aicardi-Goutieres syndrome (AGS), nonalcoholic steatohepatitis (NASH), Age-dependent macular degeneration, Myocardial infarction, Acute pancreatitis, Ischemic stroke, Sporadic aortic aneurysm and dissection, Chronic lung disease, Inflammatory bowel disease, Parkinson's disease, Traumatic brain injury or Amyotrophic lateral sclerosis (ALS).

In certain embodiments, the present description provides a method of treating a disorder (as described herein) in a subject, comprising administering to the subject identified as in need thereof, a compound of the present description. The identification of those patients who are in need of treatment for the disorders described above is well within the ability and knowledge of one skilled in the art. Certain of the methods for identification of patients which are at risk of developing the above disorders which can be treated by the subject method are appreciated in the medical arts, such as family history, and the presence of risk factors associated with the development of that disease state in the subject patient. A clinician skilled in the art can readily identify such candidate patients, by the use of, for example, clinical tests, physical examination and medical/family history.

According to another embodiment, the description provides a method of inhibiting cGAS using a composition comprising a compound of the present description or a pharmaceutically acceptable derivative thereof and a pharmaceutically acceptable carrier, adjuvant, or vehicle. In certain embodiments, a provided composition is formulated for administration to a patient in need of such composition. In some embodiments, a provided composition is formulated for oral administration to a patient.

In some embodiments, the therapeutically effective amount of a compound as defined herein can be administered to a patient alone or admixed with a pharmaceutically acceptable carrier, adjuvant, or vehicle.

The expression “pharmaceutically acceptable carrier, adjuvant, or vehicle” and equivalent expressions, refer to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions described herein may be administered orally, parenterally, by inhalation spray, topically, rectally, nasally, buccally, vaginally or via an implanted reservoir. The term “parenteral” as used herein includes subcutaneous, intravenous, intramuscular, intraarticular, intrasynovial, intrasternal, intrathecal, intrahepatic, intralesional and intracranial injection or infusion techniques. Other modes of administration also include intradermal or transdermal administration.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a provided compound, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled.

Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of the present description with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone (PVP), sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

Provided compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of the present description include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of the present description. Additionally, the description contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Pharmaceutically acceptable compositions provided herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promotors to enhance bioavailability, fluorocarbons, and/or other conventional solubilizing or dispersing agents.

Pharmaceutically acceptable compositions provided herein may be formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of provided compounds that may be combined with carrier materials to produce a composition in a single dosage form will vary depending upon the patient to be treated and the particular mode of administration. Provided compositions may be formulate such that a dosage of between 0.01-100 mg/kg body weight/day of the inhibitor can be administered to a patient receiving these compositions.

It should also be understood that a specific dosage and treatment regimen for any particular patient will depend upon a variety of factors, including age, body weight, general health, sex, diet, time of administration, rate of excretion, drug combination, the judgment of the treating physician, and the severity of the particular disease being treated. The amount of a provided compound in the composition will also depend upon the particular compound in the composition.

Compounds or compositions described herein may be administered using any amount and any route of administration effective for treating or lessening the severity of the disorders or diseases as contemplated herein. The exact amount required will vary from subject to subject, depending on the species, age, and general condition of the subject, the severity of the infection, the particular agent, its mode of administration, and the like. Provided compounds are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this disclosure can be administered to humans and other animals orally, rectally, parenterally, intracisternally, intravaginally, intraperitoneally, topically (as by powders, ointments, or drops), buccally, as an oral or nasal spray, or the like, depending on the severity of the infection being treated. In certain embodiments, provided compounds may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

In some embodiments, the composition of a compound or compounds described herein can be in combination with an additional therapeutic agent.

Provided compounds can be administered alone or in combination with one or more other therapeutic compounds, possible combination therapy taking the form of fixed combinations or the administration of a provided compound and one or more other therapeutic compounds being staggered or given independently of one another, or the combined administration of fixed combinations and one or more other therapeutic compounds. Provided compounds can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Such additional agents may be administered separately from a composition containing a provided compound, as part of a multiple dosage regimen. Alternatively, those agents may be part of a single dosage form, mixed together with a provided compound in a single composition. If administered as part of a multiple dosage regimen, the two active agents may be submitted simultaneously, sequentially or within a period of time from one another normally within five hours from one another.

Upon improvement of a subject's condition, a maintenance dose of a compound, composition or combination of the present description may be administered, if necessary. Subsequently, the dosage or frequency of administration, or both, may be reduced, as a function of the symptoms, to a level at which the improved condition is retained when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present description will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The total daily inhibitory dose of the compounds of the present description administered to a subject in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In one embodiment, treatment regimens according to the present description comprise administration to a patient in need of such treatment from about 10 mg to about 1000 mg of the compound(s) of the present description per day in single or multiple doses.

As used herein, the term “combination,” “combined,” and related terms refers to the simultaneous or sequential administration of therapeutic agents in accordance with the present description. For example, a provided compound may be administered with another therapeutic agent simultaneously or sequentially in separate unit dosage forms or together in a single unit dosage form. Accordingly, an embodiment of the present description provides a single unit dosage form comprising a provided compound, an additional therapeutic agent, and a pharmaceutically acceptable carrier, adjuvant, or vehicle for use in the methods of the present description.

The amount of both, a provided compound and additional therapeutic agent (in those compositions which comprise an additional therapeutic agent as described above) that may be combined with the carrier materials to produce a single dosage form will vary depending upon the host treated and the particular mode of administration. Preferably, compositions should be formulated such that a dosage of between 0.01-100 mg/kg body weight/day of a provided compound can be administered.

In those compositions which comprise an additional therapeutic agent, that additional therapeutic agent and the provided compound may act synergistically. Therefore, the amount of additional therapeutic agent in such compositions will be less than that required in a monotherapy utilizing only that therapeutic agent. In such compositions a dosage of between 0.01-1,000 g/kg body weight/day of the additional therapeutic agent can be administered.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In another aspect, the present description provides a method of method of synthesizing a compound of any of the formulae herein. Another embodiment is a method of making a compound of any of the formulae herein using anyone, or combination of, reactions delineated herein. The method can include the use of one or more intermediates or chemical reagents delineated herein.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### EXAMPLES

The Examples set forth herein below provide syntheses and experimental results obtained for certain exemplary compounds. Unless otherwise indicated, all numbers expressing quantities of ingredients, reaction conditions, concentrations, properties, stabilities, and so forth used in the specification and claims are to be understood as being modified in all instances by the term “about.” At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

The following is to be construed as merely illustrative, and not limitations of the preceding disclosure in any way whatsoever. Those skilled in the art will promptly recognize appropriate variations from the procedures both as to reactants and as to reaction conditions and techniques. In some cases, starting materials or intermediates may be commercially available.

### Example 1: Chemical Synthesis of Exemplary Compounds

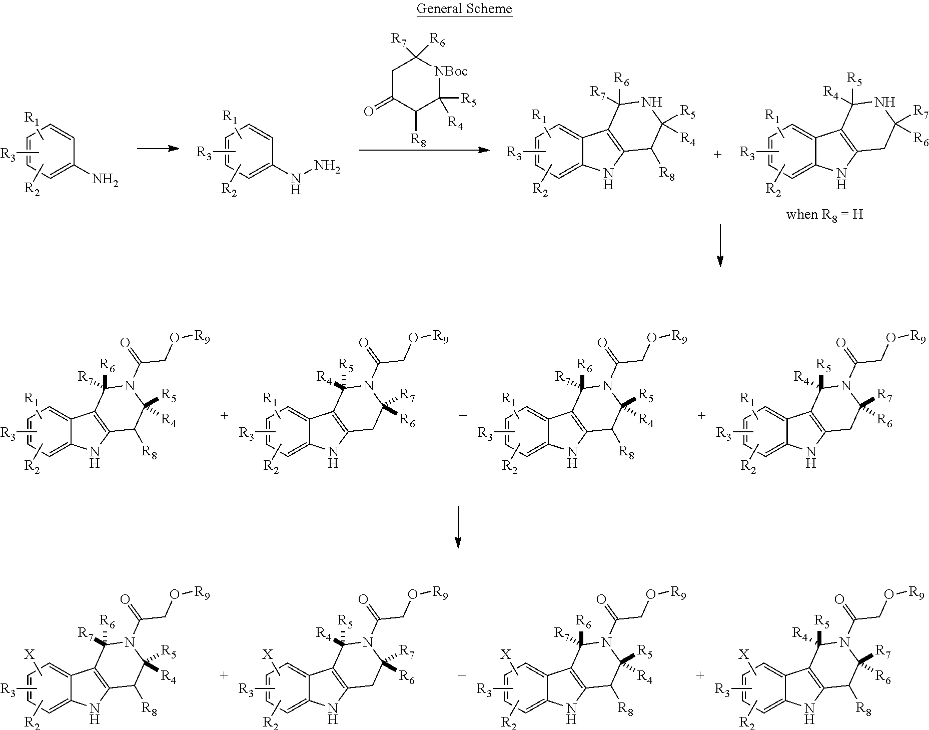

As used herein, the following abbreviations may have the following meanings:

| Abbreviation | Term |
|---|---|
| AcOH | Acetic acid |
| ATP | Adenosine triphosphate |
| DCM | Dichloromethane |
| DIPEA | N,N-Diisopropylethylamine |
| DMF | N,N-dimethyl formamide |
| DMSO | Dimethylsulfoxide |
| dsDNA | double stranded DNA |
| DTT | Dithiothreitol |
| equiv | equivalent |
| EtOAc or EA | Ethyl acetate |
| GTP | Guanosine triphosphate |
| h | Hour(s) |
| h-cGAS | Human-cGAS |
| HATU | 1-[Bis(dimethylamino)methylene]-1H-1,2,3-triazolo[4,5- |
| | b]pyridinium 3-oxid hexafluorophosphate |
| Prep-HPLC | Preparative High-performance |
| | liquid chromatography |
| LC-MS (ESI) | Liquid Chromatography-Mass |
| | Spectrometry (Electrospray Ionization) |
| min | Minute(s) |
| NBS | N-Bromosuccinimide |
| PE | Petroleum ether |
| Pd(dppf)Cl₂ | [1,1′- |
| | Bis(diphenylphosphino)ferrocene]dichloropalladium(II) |
| r.t. | room temperature |
| Xantphos | 4,5-Bis(diphenylphosphino)-9,9-dimethylxanthene |
| TMSOK | Trimethylsilanol potassium salt |
| Tris-HCl | Tris(hydroxymethyl)aminomethane hydrochloride |

### Example 1A

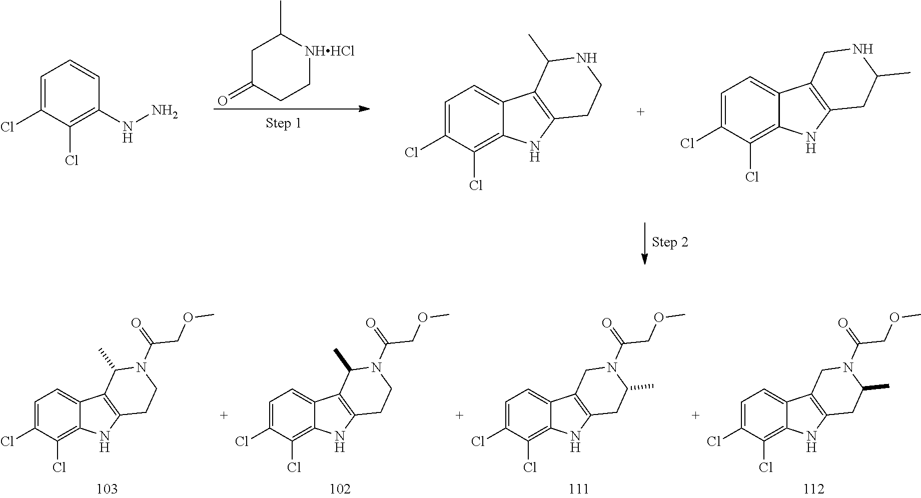

### Step 1

A solution of (2,3-Dichlorophenyl)hydrazine hydrochloride (1:1) (1.00 eq, 1400 mg, 6.43 mmol) and rac-(2R)-2-methylpiperidin-4-one hydrochloride (1.00 eq, 962 mg, 6.43 mmol) in ethanol (10 mL) was treated with sulfuric acid (10.0 eq, 3.6 mL, 64.3 mmol) and stirred at 100° C. for 8 h by microwave. The volatiles were removed under pressure. The residue was purified using reverse phase chromatography eluting with 0-100% MeCN/H₂O to afford the desired product, which was a mixture of regioisomers and enantiomers.

### Step 2

A solution of a mixture of rac-6,7-dichloro-3-methyl-2,3,4,5-tetrahydro-1H-pyrido[4,3-b]indole and rac-6,7-dichloro-1-methyl-2,3,4,5-tetrahydro-1H-pyrido[4,3-b]indole (1.00 eq, 380 mg, 1.30 mmol) and 2-methoxyacetic acid (2.00 eq, 0.021 mL, 2.61 mmol) in 1 mL DMF was treated with DIPEA (3.00 eq, 0.68 mL, 3.91 mmol) and HATU (1.10 eq, 545 mg, 1.43 mmol) and stirred at r.t. for 20 min. The residue was purified using reverse phase chromatography eluting with 0-100% MeCN/H₂O to afford the desired products.

### Example 1B

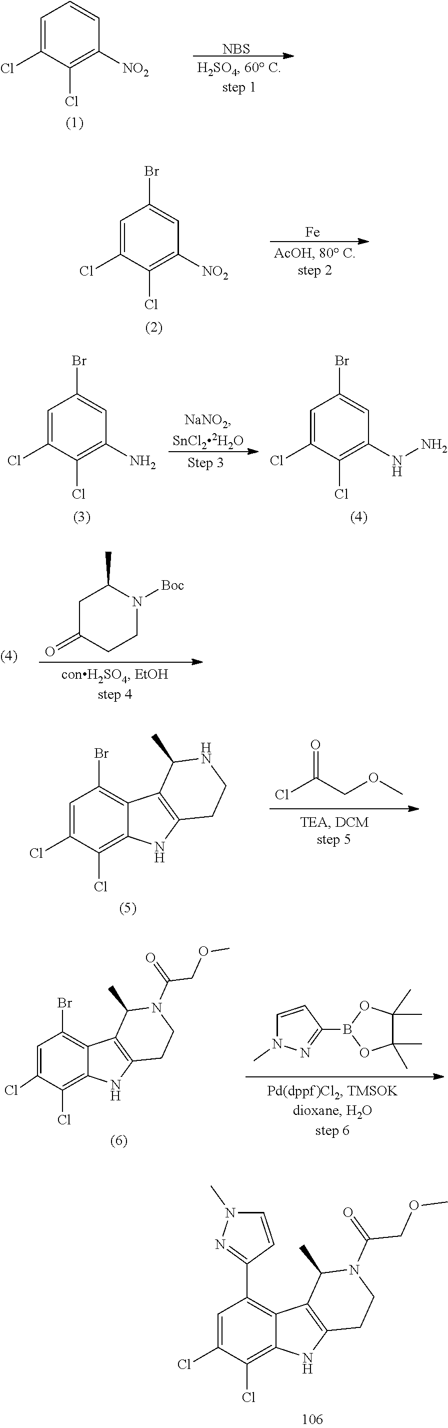

### Step 1

A suspension of 2,3-dichloronitrobenzene (2.00 g, 10.417 mmol, 1.00 equiv) and NBS (2.22 g, 0.012 mmol, 1.2 equiv) in H₂SO₄ (15.00 mL) was stirred for 4 h at 60° C. in an oil bath. And then the reaction was then quenched by the addition of 200 mL of water/ice, extracted with 3×50 mL of ethyl acetate and the organic layers combined. The organic layer was washed with 1×50 mL of brine, dried over anhydrous sodium sulfate and concentrated under vacuum. The residue was applied onto a silica gel column eluting with ethyl acetate/petroleum ether (1:15) to afford 1.47 g (52.09%) of 5-bromo-1,2-dichloro-3-nitrobenzene as a light yellow solid. LC-MS (ESI) [M+H]⁺=270/272/274.

### Step 2

A solution of 5-bromo-1,2-dichloro-3-nitrobenzene (2.00 g, 7.383 mmol, 1.00 equiv) and Fe (1.65 g, 29.532 mmol, 4.00 equiv) in AcOH (15.00 mL) was stirred for 1.5 h at 80° C. The reaction was quenched by the addition of Water (50 mL) at room temperature. The resulting mixture was extracted with EtOAc (3×40 mL). The combined organic layers were washed with brine (1×10 mL), dried over anhydrous Na₂SO₄. After filtration, the filtrate was concentrated under reduced pressure. The residue was purified by silica gel column chromatography eluting with PE/EtOAc (9:1) to afford 5-bromo-2,3-dichloroaniline (1.6228 g, 91.24%) as a crude yellow oil. LC-MS (ESI) [M+H]⁺=240/242/244.

### Step 3

A suspension of 5-bromo-2,3-dichloroaniline (1.00 g, 4.151 mmol, 1.00 equiv) in H₂O (3.00 mL) was added HCl (3.00 mL, 12M) and NaNO₂ (661.00 mg, 9.580 mmol, 2.31 equiv) at 0° C. The resulting solution was stirred for 4 h at 0° C. in a water/ice bath and a solution of SnCl₂.2H₂O (2.71 g, 12.010 mmol, 2.89 equiv) in HCl (3.00 mL, 12M) was added. The resulting solution was reacted for another 12 h at 0° C. The solids were collected by filtration, washed with 3×10 mL of EA. This resulted in 1.2 g of (5-bromo-2,3-dichlorophenyl)hydrazine as a light yellow solid. LC-MS (ESI) [M+H]⁺=255/257/259.

### Step 4

A suspension of (5-bromo-2,3-dichlorophenyl)hydrazine (400.00 mg, 1.563 mmol, 1.00 equiv) and tert-butyl (R)-2-methyl-4-oxopiperidine-1-carboxylate (680.0 mg, 3.193 mmol, 2.0 equiv) in Dioxane (2.00 mL) was added H₂SO₄ (1.54 g, 15.702 mmol, 10.05 equiv) and the solution was stirred for 12 h at 110° C. in an oil bath. The reaction was quenched by the addition of 5 mL of water, and then the pH value of the solution was adjusted to 7 with NaOH (10 mol/L). The resulting solution was extracted with 5×10 mL of ethyl acetate and the aqueous layers combined and concentrated under vacuum, and then the crude product was purified by C18 reverse phase eluting with MeCN/H₂O to afford (R)-9-bromo-6,7-dichloro-1-methyl-2,3,4,5-tetrahydro-1H-pyrido[4,3-b]indole as a light yellow solid. LC-MS (m/z) [M+H]⁺=333/335/337.

### Step 5

A suspension of (R)-9-bromo-6,7-dichloro-1-methyl-2,3,4,5-tetrahydro-1H-pyrido[4,3-b]indole (370.00 mg, 1.1 mmol, 1.00 equiv) in DCM was added NEt₃ (222.6 mg, 2.2 mmol, 2.0 equiv) and 2-methoxyacetyl chloride (143.2 mg, 1.32 mmol, 1.2 equiv). The resulting solution was stirred for 30 min at room temperature, and then the reaction was then quenched by the addition of MeOH. The resulting mixture was concentrated under vacuum to afford (R)-1-(9-bromo-6,7-dichloro-1-methyl-1,3,4,5-tetrahydro-2H-pyrido[4,3-b]indol-2-yl)-2-methoxyethan-1-one. LC-MS (ESI) [M+H]+=405/407.

### Step 6

A suspension of (R)-1-(9-bromo-6,7-dichloro-1-methyl-1,3,4,5-tetrahydro-2H-pyrido[4,3-b]indol-2-yl)-2-methoxyethan-1-one (320 mg, 0.788 mmol, 1.0 equiv), Pd(dppf)Cl₂ (35.00 mg, 0.048 mmol, 0.20 equiv), xantphos (55.00 mg, 0.095 mmol, 0.20 equiv), TMSOK (2.5 equiv) and 1-methyl-3-(4,4,5,5-tetramethyl-1,3,2-dioxaborolan-2-yl)-1H-pyrazole (409.8 mg, 1.97 mmol, 2.5 equiv) in dioxane (10 mL) and H₂O (2 mL) was stirred for 6 h at 80° C. in an oil bath. The solids were filtered out and the resulting mixture was concentrated under vacuum. The crude product was purified by chiral Prep-HPLC to afford (R)-1-(6,7-dichloro-1-methyl-9-(1-methyl-1H-pyrazol-3-yl)-1,3,4,5-tetrahydro-2H-pyrido[4,3-b]indol-2-yl)-2-methoxyethan-1-one and (R)-1-(6,7-dichloro-3-methyl-9-(1-methyl-1H-pyrazol-3-yl)-1,3,4,5-tetrahydro-2H-pyrido[4,3-b]indol-2-yl)-2-methoxyethan-1-one. LC-MS (ESI) [M+H]⁺=407/409.

### Example 2: Biological Activity—h-cGAS Kinase-Glo Assays

Certain compounds of the present disclosure were tested for their h-cGAS inhibition activity using the methodology reported in Lama et al., “Development of human cGAS-specific small-molecule inhibitors for repression of dsDNA-triggered interferon expression”, Nature Communications 10, Article number: 2261 (2019), with slight changes to some conditions as shown in Table 2. The results of the assays, expressed as IC₅₀ values, are reported in Table 3.

TABLE 2
Summary of assay conditions

| | Lama et al. 2019 | Present Disclosure |
|---|---|---|
| Enzyme | | |
| h-cGAS (nM) | 100 | 40 |
| Buffer | | |
| Tris-HCl pH 7.4 (mM) | 20 | 20 |
| MgCl₂ (mM) | 5 | 10 |
| NaCl (mM) | 150 | 25 |
| Tween ™-20 (%) | 0.01 | 0.01 |
| ZnCl₂ | 1 | 1 |
| DTT (mM) | 1 | 1 |
| DMSO (%) | 0.5 | 5 |
| Substrates | | |
| ATP (uM) | 100 | 100 |
| GTP (uM) | 100 | 100 |
| dsDNA (nM) | 25 | 25 |
| Assay | | |
| Plate (wells) | 384 | 384 |
| Incubation length (h) | 7 | 2 |
| Total volume (μl) | 20 | 20 |
| Kinase-Glo Max (μl) | 20 | 20 |

TABLE 3
Assay results

| Com- | | IC50 |
|---|---|---|
| pound | | h-cGAS |
| number | Compound structure | (μM) |
| 102 | 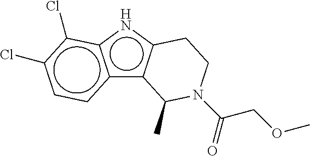 | 0.02717 |
| 103 | 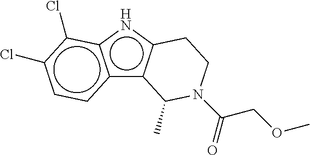 | 4.326 |
| 104 | 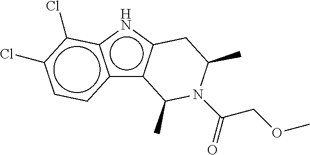 | 0.05172 |
| 105 | 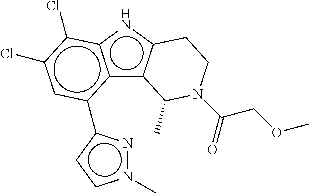 | 3.823 |
| 106 | 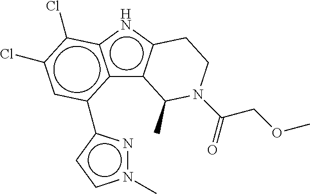 | 4.546 |
| 107 | 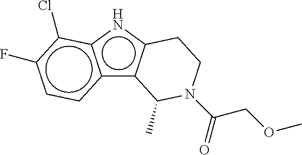 | 0.05179 |
| 108 | 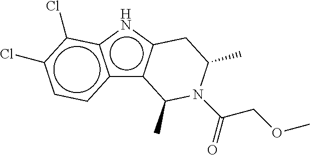 | 0.06232 |
| 109 | 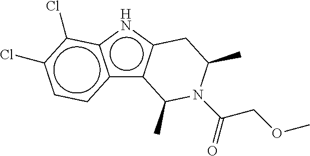 | 0.01512 |
| 110 | 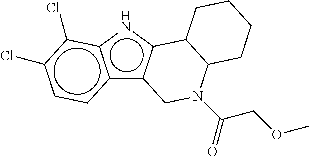 | 2.228 |
| 111 | 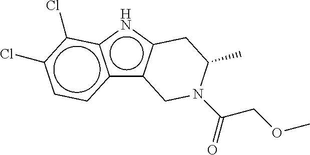 | 0.4006 |
| 112 | 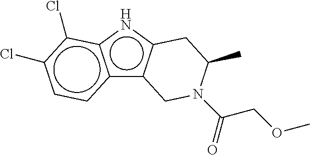 | 0.2363 |
| 113 | 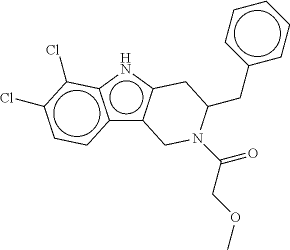 | 4.231 |
| 114 | 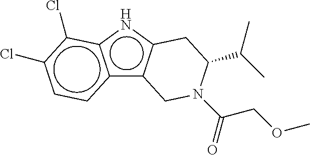 | 0.7179 |
| 115 | 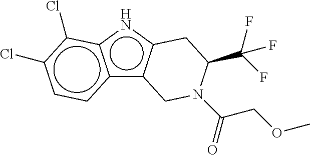 | 1.382 |
| 116 | 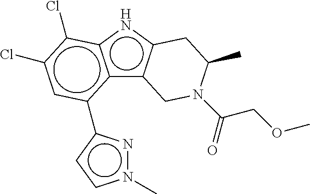 | 0.157 |
| 117 | 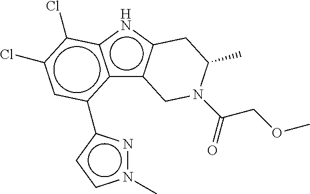 | 0.2211 |
| 118 | 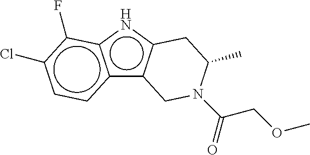 | 0.3159 |
| 119 | 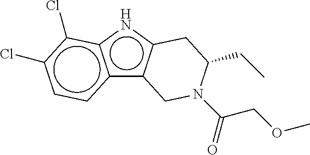 | 0.4701 |
| 120 | 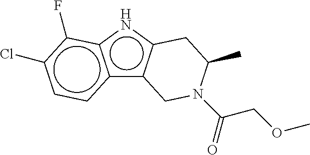 | 0.3611 |
| 121 | 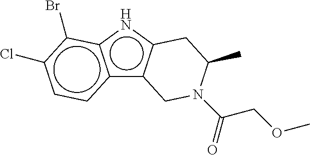 | 7.632 |
| 122 | 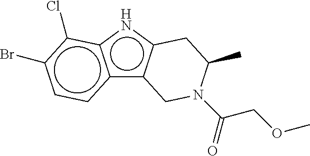 | 0.205 |
| 123 | 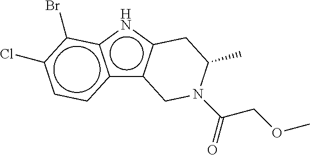 | 0.1332 |
| 124 | 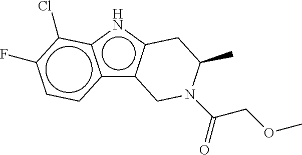 | 1.333 |
| 125 | 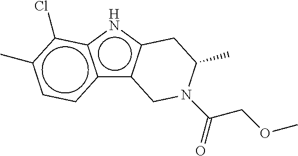 | 0.7563 |
| 126 | 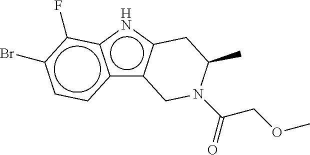 | 0.3869 |

Although the invention has been illustrated and described with respect to one or more implementations, equivalent alterations and modifications will occur to others skilled in the art upon the reading and understanding of this specification. In addition, while a particular feature of the invention may have been disclosed with respect to only one of several implementations, such feature may be combined with one or more other features of the other implementations as may be desired and advantageous for any given or particular application.

Accordingly, it is understood that the examples and embodiments described herein are for illustrative purposes only and that various modifications or changes in light thereof will be suggested to persons skilled in the art and are to be included within the spirit and purview of this application and scope of the appended claims. Any publication, document, patent, patent application or publication referred to herein should be construed as incorporated by reference each in their entirety for all purposes.

1. A compound of formula (I): 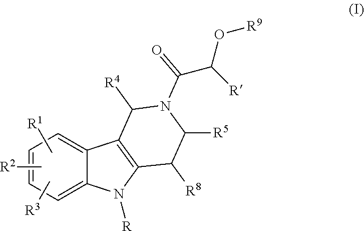
or a pharmaceutically acceptable salt, solvate, ester or prodrug thereof,
wherein:
R¹, R² and R³ independently represent hydrogen, halogen, CN, unsubstituted or substituted C₁-C₆ alkoxy, unsubstituted or substituted C₂-C₆ alkenyl, unsubstituted or substituted linear or branched C₁-C₆ alkyl, unsubstituted or substituted C₃-C₆ cycloalkyl or cycloalkenyl, unsubstituted or substituted C₆ aryl, unsubstituted or substituted 4 to 10 membered heterocycloalkyl being saturated or partially unsaturated, unsubstituted or substituted 4 to 10 membered heteroaryl, —NR¹⁰C(═O)R¹¹, —C(═O)NR¹²R¹³ or —CHR¹⁴R¹⁵, with at least one of R¹, R² and R³ being different than hydrogen;
R⁴, R⁵ and R⁸ independently represent H, unsubstituted or substituted linear or branched C₁-C₆ alkyl, —CH₂Ph, with at least one of R⁴ and R⁵ being different than hydrogen;
or
R⁵ and R⁸ are linked together to form a C₅-C₆ cycloalkyl;
R and R′ independently represent H or linear or branched C₁-C₃alkyl;
R⁹ is H or linear or branched C₁-C₃alkyl;
R¹⁰ is H or linear or branched C₁-C₃alkyl;
R¹¹ is unsubstituted or substituted linear or branched C₁-C₃alkyl;
R¹² is H or linear or branched C₁-C₃alkyl;
R¹³ is linear or branched C₁-C₃alkyl;
R¹⁴ is H or linear or branched C₁-C₃alkyl;
R¹⁵ is unsubstituted or substituted C₆ aryl, unsubstituted or substituted C₁-C₃ cycloalkyl or unsubstituted or substituted 5 to 6 membered heterocycloalkyl;
wherein the heterocycloalkyl and heteroaryl groups include 1 to 3 heteroatoms independently selected from the group consisting of N, O and S;
wherein, when any alkyl, alkoxy, alkenyl, cycloalkyl, cycloalkenyl, saturated or partially unsaturated heterocycloalkyl, aryl or heteroaryl groups are substituted, these groups are independently substituted with 1 to 3 substituents selected from the group consisting of halogen, hydroxy, methoxy, methyl, oxo (═O), CN, —NH₂, —NH(C₁-C₃alkyl), —N(C₁-C₃alkyl)₂, —NH(CO)CF₃, —CH₂OH, —CF₃, —CHF₂, —CH₂F, —SO₂NH(CH₂)₃OH and pyrazolyl.

2. The compound of claim 1 or a pharmaceutically acceptable salt, solvate, ester or prodrug thereof, wherein the compound is of formula (Ia) or (Ib)

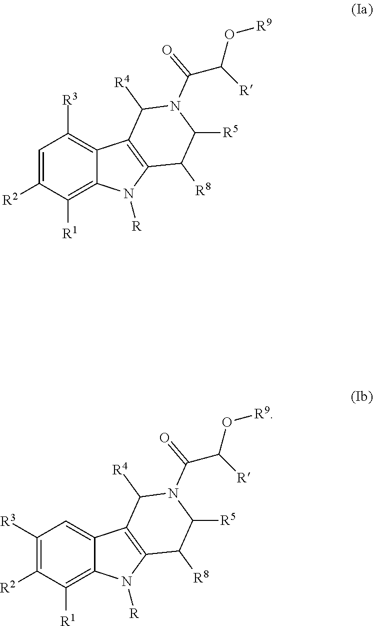

3. The compound of claim 2 or a pharmaceutically acceptable salt, solvate, ester or prodrug thereof, wherein the compound is of formula (Ia).

4. The compound of any one of claims 1 to 3, wherein R¹ and R² are both different than hydrogen.

5. The compound of any one of claims 1 to 4, wherein R¹ and R² independently represent halogen, CN, unsubstituted or substituted C₁-C₆ alkoxy, unsubstituted or substituted C₂-C₆ alkenyl, or unsubstituted or substituted linear or branched C₁-C₆ alkyl, wherein, when any alkyl, alkoxy, or alkenyl groups are substituted, these groups are independently substituted with 1 to 3 halogen atoms.

6. The compound of any one of claims 1 to 5, wherein R¹ and R² independently represent halogen, CN, unsubstituted C₁-C₂ alkoxy, C₁-C₂ alkoxy substituted with 1 to 3 halogen atoms, unsubstituted C₁-C₂ alkyl, C₁-C₂ alkyl substituted with 1 to 3 halogen atoms, or unsubstituted C₂-C₃ alkenyl.

7. The compound of any one of claims 1 to 6, wherein R¹ and R² independently represent halogen or methyl.

8. The compound of any one of claims 1 to 7, wherein R¹ and R² both represent halogen.

9. The compound of any one of claims 1 to 8, wherein R³ represents hydrogen, halogen, CN, —NR¹⁰C(═O)R¹¹, or —C(═O)NR¹²R¹³ with R¹⁰ and R¹² represent hydrogen and R¹¹ and R¹³ represent —CH₃, unsubstituted C₁-C₃ alkoxy, unsubstituted C₂-C₃ alkenyl, unsubstituted C₁-C₃ alkyl, or C₁-C₃ alkyl substituted with 1 to 3 groups selected from halogen, methoxy, CN, —NH₂, —NH(C₁-C₃alkyl), and —N(C₁-C₃alkyl)₂,
or R³ is

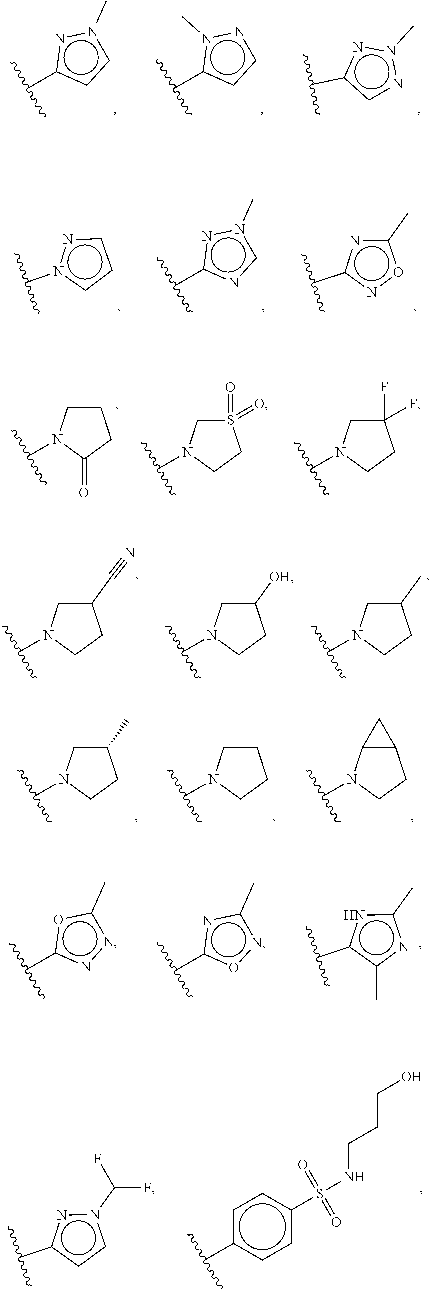

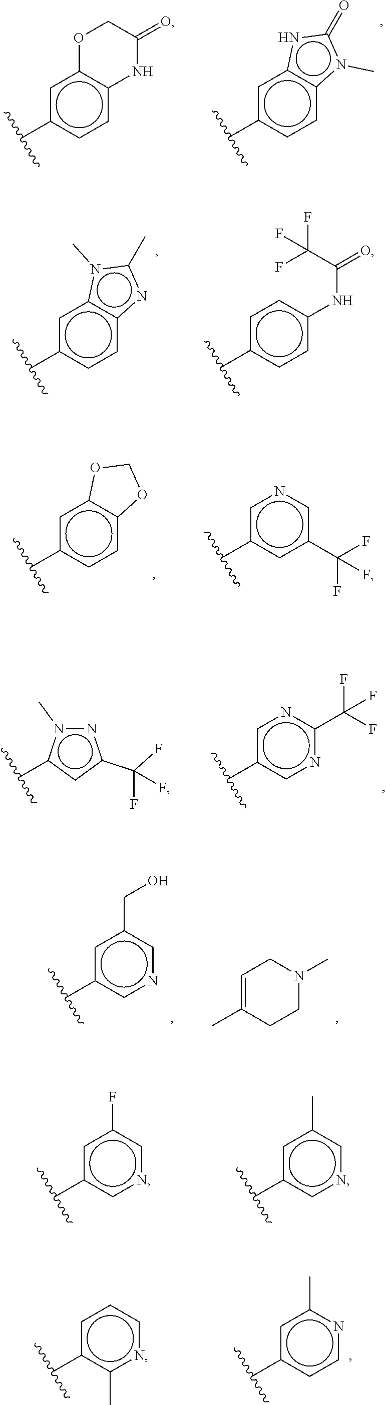

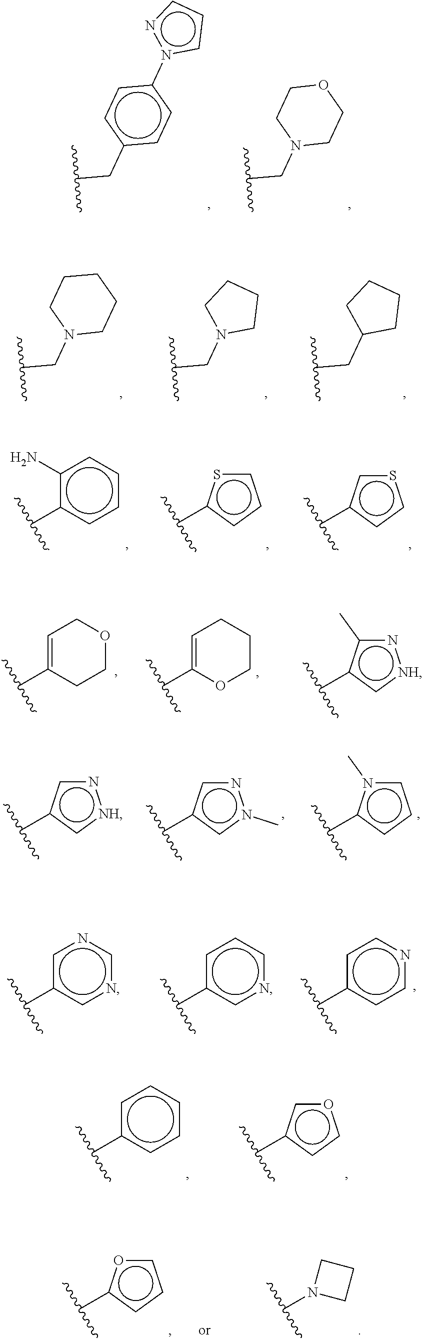

10. The compound of any one of claims 1 to 9, wherein R³ represents hydrogen, halogen, CN, —CH₃, —OCH₃, —CH═CH₂, —CH₂CH₂OCH₃, —CH₂N(CH)₂, —NR¹⁰C(═O)R¹¹, or —C(═O)NR¹²R¹³ with R¹⁰ and R¹² represent hydrogen and R¹¹ and R¹³ represent —CH₃,
or R³ is

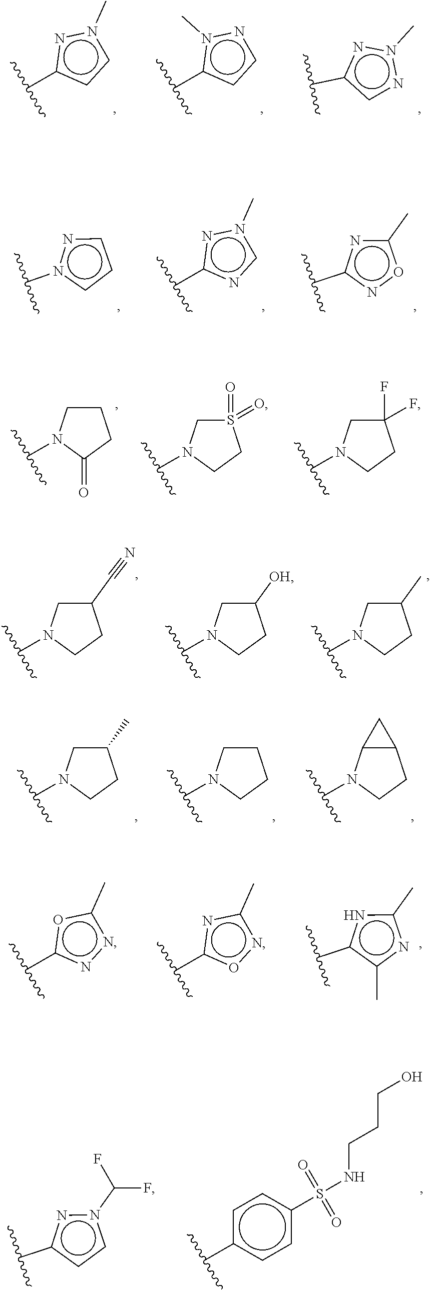

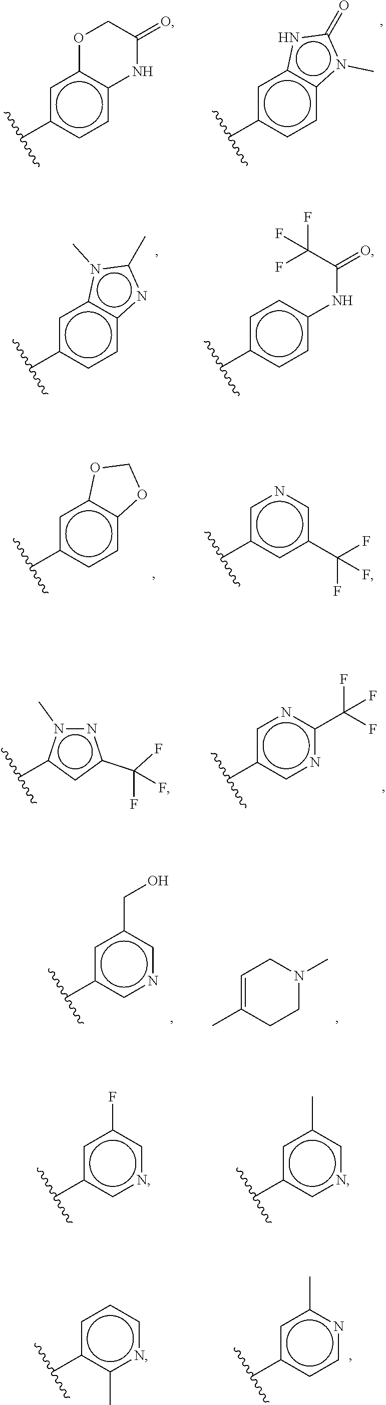

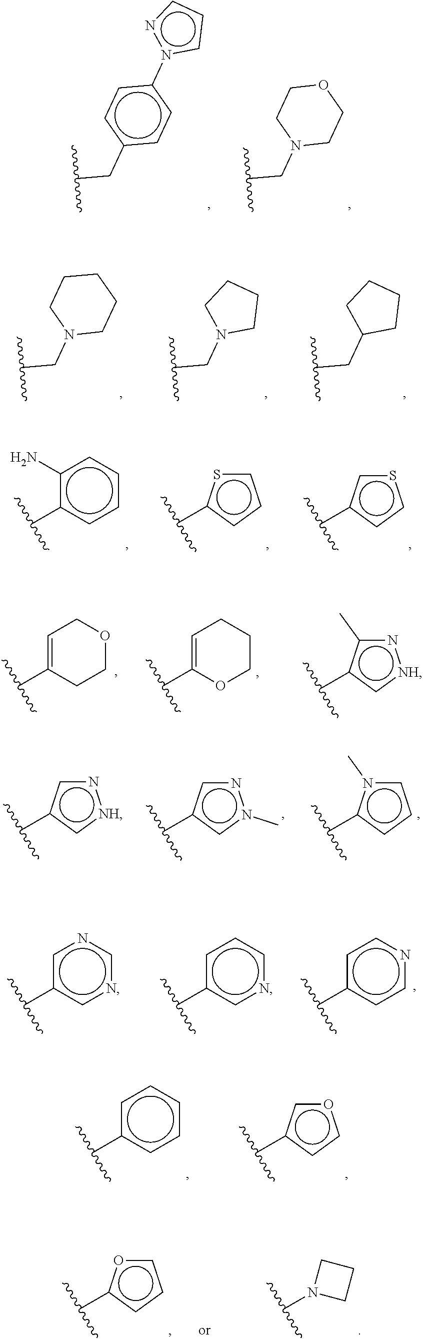

11. The compound of any one of claims 1 to 10, wherein R³ represents hydrogen or

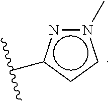

12. The compound of any one of claims 1 to 11, wherein R¹ and R² represent halogen or methyl and R³ represents hydrogen.

13. The compound of any one of claims 1 to 12, wherein R is hydrogen.

14. The compound of any one of claims 1 to 13, wherein R⁴, R⁵ and R⁸ independently represent H, unsubstituted or substituted linear or branched C₁-C₃ alkyl, or —CH₂Ph, with at least one of R⁴ and R⁵ being different than hydrogen; or R⁵ and R⁸ are linked together to form a C₅-C₆ cycloalkyl; and when the C₁-C₃ alkyl is substituted, the substituent(s) is(are) halogen.

15. The compound of any one of claims 1 to 14, wherein R⁴, R⁵ and R⁸ independently represent H, —CH₃, —CH₂CH₃, —CH(CH₃)₂, —CF₃, or —CH₂Ph, with at least one of R⁴ and R⁵ being different than hydrogen; or R⁵ and R⁸ are linked together to form a C₆ cycloalkyl and R⁴ represents hydrogen.

16. The compound of any one of claims 1 to 15, wherein at least one of R⁴ and R⁵ is —CH₃.

17. The compound of any one of claims 1 to 16, wherein R′ represents hydrogen.

18. The compound of any one of claims 1 to 17, wherein R⁹ represents hydrogen or methyl.

19. The compound of any one of claims 1 to 17, wherein R⁹ represents hydrogen.

20. The compound of any one of claims 1 to 17, wherein R⁹ represents methyl.

21. The compound of claim 1 or a pharmaceutically acceptable salt, solvate, ester or prodrug thereof, wherein the compound is of formula (Ic)

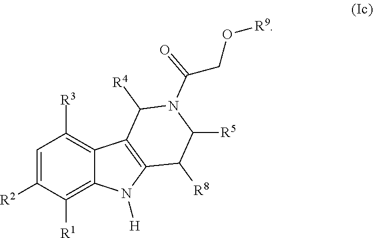

22. The compound of claim 21, wherein R¹ and R² are as defined in any one of claims 4 to 8.

23. The compound of claim 21 or 22, wherein R³ is as defined in any one of claims 9 to 11.

24. The compound of claim 21, wherein R¹ and R² independently represent halogen or methyl and R³ represents hydrogen.

25. The compound of claim 21, wherein R¹ represents halogen, R² represents halogen or methyl, and R³ represents hydrogen.

26. The compound of any one of claims 21 to 25, wherein R⁴, R⁵ and R⁸ independently represent H, —CH₃, —CH₂CH₃, —CH(CH₃)₂, —CF₃, or —CH₂Ph, with at least one of R⁴ and R⁵ being different than hydrogen; or R⁵ and R⁸ are linked together to form a C₆ cycloalkyl and R⁴ represents hydrogen.

27. The compound of any one of claims 21 to 26, wherein R⁹ represents hydrogen or methyl.

28. The compound of any one of claims 21 to 27, wherein R⁹ represents methyl.

29. The compound of claim 1 or a pharmaceutically acceptable salt, solvate, ester or prodrug thereof, wherein the compound is selected from Compounds 1 to 140 of Table 1.

30. The compound of claim 1 or a pharmaceutically acceptable salt, solvate, ester or prodrug thereof, wherein the compound is selected from Compounds 102 to 126 of Table 1.

31. A pharmaceutical composition, comprising a compound of any one of claims 1 to 30, together with a pharmaceutically acceptable carrier, diluent or excipient.

32. Use of a compound according to any one of claims 1 to 30 for the treatment of a disease or condition for which a cGAS inhibitor is indicated.

33. The use according to claim 32, wherein the disease or condition is autoinflammation or an autoimmune disease such as systemic lupus erythematosus (SLE), Aicardi-Goutieres syndrome (AGS), nonalcoholic steatohepatitis (NASH), Age-dependent macular degeneration, Myocardial infarction, Acute pancreatitis, Ischemic stroke, Sporadic aortic aneurysm and dissection, Chronic lung disease, Inflammatory bowel disease, Parkinson's disease, Traumatic brain injury or Amyotrophic lateral sclerosis (ALS).

34. Use of a compound according to any one of claims 1 to 30 in the manufacture of a medicament for the treatment of a disease or condition for which a cGAS inhibitor is indicated.

35. The use according to claim 34, wherein the disease or condition is autoinflammation or an autoimmune disease such as systemic lupus erythematosus (SLE), Aicardi-Goutieres syndrome (AGS), nonalcoholic steatohepatitis (NASH), Age-dependent macular degeneration, Myocardial infarction, Acute pancreatitis, Ischemic stroke, Sporadic aortic aneurysm and dissection, Chronic lung disease, Inflammatory bowel disease, Parkinson's disease, Traumatic brain injury or Amyotrophic lateral sclerosis (ALS).

36. Use of a compound according to any one of claims 1 to 30 for the treatment of a disease or condition selected from the group consisting of Aicardi-Goutieres syndrome (AGS), systemic lupus erythematosus (SLE), nonalcoholic steatohepatitis (NASH), Age-dependent macular degeneration, Myocardial infarction, Acute pancreatitis, Ischemic stroke, Sporadic aortic aneurysm and dissection, Chronic lung disease, Inflammatory bowel disease, Parkinson's disease, Traumatic brain injury and Amyotrophic lateral sclerosis (ALS).

37. A method for treating a disease or condition for which a cGAS inhibitor is indicated, which comprises administering to a subject in need thereof, a therapeutically effective amount of a compound according to any one of claims 1 to 30.

38. The method according to claim 37, wherein the disease or condition is autoinflammation or an autoimmune disease, such as systemic lupus erythematosus (SLE), Aicardi-Goutieres syndrome (AGS), nonalcoholic steatohepatitis (NASH), Age-dependent macular degeneration, Myocardial infarction, Acute pancreatitis, Ischemic stroke, Sporadic aortic aneurysm and dissection, Chronic lung disease, Inflammatory bowel disease, Parkinson's disease, Traumatic brain injury or Amyotrophic lateral sclerosis (ALS).

39. A method for the treatment of a disease or condition selected from autoinflammation and an autoimmune disease, such as systemic lupus erythematosus (SLE), Aicardi-Goutieres syndrome (AGS), nonalcoholic steatohepatitis (NASH), Age-dependent macular degeneration, Myocardial infarction, Acute pancreatitis, Ischemic stroke, Sporadic aortic aneurysm and dissection, Chronic lung disease, Inflammatory bowel disease, Parkinson's disease, Traumatic brain injury or Amyotrophic lateral sclerosis (ALS), which comprises administering to a subject in need thereof, a therapeutically effective amount of a compound according to any one of claims 1 to 30.
